(19) United States
(12) Patent Application Publication
(10) Pub. No.: US 2022/0270423 A1
(43) Pub. Date: Aug. 25, 2022

(54) IDENTITY-BASED ENABLEMENT OF EVENT ACCESS CONTROL

(71) Applicant: Alan Mark Gelfand, Vancouver (CA)

(72) Inventor: Alan Mark Gelfand, Vancouver (CA)

(21) Appl. No.: 17/675,853

(22) Filed: Feb. 18, 2022

Related U.S. Application Data
(60) Provisional application No. 63/151,684, filed on Feb. 20, 2021.

(51) Int. Cl. G07C 9/25; G16H 10/60

(52) U.S. Cl. CPC G07C 9/25; G16H 10/60

(57) ABSTRACT

Methods, systems, and techniques for identifying attendees at live events using biometric information, and for conditioning event admittance based on satisfaction of one or more health criteria.

### CROSS-REFERENCE TO RELATED APPLICATIONS

This application claims the benefit of U.S. provisional patent application Ser. No. 63/151,684, filed Feb. 20, 2021 and entitled “Systems and Methods for Identifying Attendees at Live Events,” the disclosure of which is hereby incorporated herein by reference in its entirety.

### TECHNICAL FIELD

The present disclosure is directed at methods, systems, and techniques for identifying attendees at live events, such as concerts, sports events, plays, skiing and other recreational activities, conferences, and exhibitions

### BACKGROUND

Live events commonly have thousands of attendees. Event hosts can benefit from knowing the identities of attendees at their events. Often, tickets to an event are bearer instruments in which any person holding the ticket, regardless of identity, is entitled entry to the event. This is not conducive to knowing the identities of all event attendees. Even in situations where a ticket is labeled with the name of a particular ticketholder and intended for use only by that ticketholder, conventionally ticketholder identity is manually verified immediately prior to event admittance. This scales poorly to large crowd sizes and requires significant extra expense (e.g., labor for extra checkpoints), consequently resulting in delays for attendees and additional security risks (e.g., due to increased crowding in an uncontrolled environment prior to the transition to a controlled environment). Even when workable from a logistical perspective, a manual system is vulnerable to circumvention by persons who wish to avoid having their identities known.

Additionally, the effect of communicable diseases, such as COVID-19, on public health and consequently the economy are becoming increasingly well understood. Certain diseases, which can be spread in aerosol form or via droplets, are spread much more readily when people are crowded together than when they are separated from each other by significant physical distance. Those diseases also typically spread more easily when people are gathered indoors or congregated outdoors. While manual health status checks are becoming more common, they are also more prone to error than automated scans, which governments are starting to mandate with greater frequency. Knowing the identities of persons entering an event and their current health status can be useful for safety and security reasons as well.

### SUMMARY

According to a another aspect, there is provided a method comprising: confirming, by one or more servers and in advance of an event, that a ticketholder of the event satisfies one or more health criteria; after confirming that the ticketholder satisfies the one or more health criteria: reading current identification information, at a location away from an entrance of an event, that identifies the ticketholder; after the current identification information is read, communicating the current identification information to the one or more servers; reading ticket information from a ticket held by the ticketholder at the entrance of the event; after the ticket information is read, communicating the ticket information to the one or more servers; and determining that the ticketholder may be granted access to the event after determining that: the ticket information is valid; and the current identification information matches registered identification information registered with the one or more servers before the current identification information is read.

According to another aspect, there is provided a system comprising: a identification validation unit for placement at an event away from an entrance of the event, the identification validation unit comprising a first processor, an identification reader that is communicative with the first processor, and a first computer readable medium that is communicative with the first processor and that comprises instructions that when executed by the first processor cause the identification unit to read current identification information from identification presented to the valued identification reader and to communicate the current identification information to one or more servers; a ticket validation unit for placement at the entrance of the event, the ticket validation unit comprising a second processor, a ticket reader that is communicative with the second processor, and a second computer readable medium that is communicative with the second processor and that comprises instructions that when executed by the second processor cause the ticket validation unit to read ticket information from a ticket presented to the ticket reader and to communicate the ticket information to the one or more servers; wherein the one or more servers are configured to: confirm that a ticketholder of the event satisfies one or more health criteria; and after confirming that the ticketholder satisfies the one or more health criteria: receive the current identification information from the identification validation unit; receive the ticket information from the ticket validation unit; and determine that the ticketholder may be granted access to the event after determining that: the ticket information is valid; and the current identification information matches registered identification information registered with the one or more servers before the current identification information is read.

According to another aspect, there is provided a method comprising: obtaining a first picture of a face of a ticketholder; obtaining a reference photo identification of the ticketholder, wherein the reference photo identification comprises a second picture of the ticketholder and confirms an identity of the ticketholder; confirming that the first picture and the second picture are both of the ticketholder; generating an identification record linking the ticketholder to the identity confirmed by the reference photo identification; and storing the identification record in a database.

According to another aspect, there is provided a method comprising: reading current identification information, at a location away from an entrance of an event, that identifies the ticketholder, wherein the current identification information comprises biometric facial information of the ticketholder; after the current identification information is read, communicating the current identification information to the one or more servers; reading ticket information from a ticket held by the ticketholder at the entrance of the event; after the ticket information is read, communicating the ticket information to the one or more servers; and determining that the ticketholder may be granted access to the event after determining that: the ticket information is valid; and the current identification information matches registered identification information registered with the one or more servers before the current identification information is read.

According to another aspect, there is provided a system comprising: a identification validation unit for placement at an event away from an entrance of the event, the identification validation unit comprising a first processor, an identification reader that is communicative with the first processor, and a first computer readable medium that is communicative with the first processor and that comprises instructions that when executed by the first processor cause the identification unit to read current identification information from identification presented to the valued identification reader and to communicate the current identification information to one or more servers; a ticket validation unit for placement at the entrance of the event, the ticket validation unit comprising a second processor, a ticket reader that is communicative with the second processor, and a second computer readable medium that is communicative with the second processor and that comprises instructions that when executed by the second processor cause the ticket validation unit to read ticket information from a ticket presented to the ticket reader and to communicate the ticket information to the one or more servers; wherein the one or more servers are configured to: receive the current identification information from the identification validation unit, wherein the current identification information comprises biometric facial information of the ticketholder; receive the ticket information from the ticket validation unit; and determine that the ticketholder may be granted access to the event after determining that: the ticket information is valid; and the current identification information matches registered identification information registered with the one or more servers before the current identification information is read.

According to an aspect there is provided a method for identifying an event attendee comprising: reading biometric identification information from one or more value or biometric identifications at one or more locations or via mobile devices at or near an event away from the entrance of the event and communicating the read biometric identification information to one or multiple servers on and off location; reading ticket information from a ticket at the entrance of the event and communicating the ticket information to the server; determining by the server whether to grant access to the event to a ticketholder of the ticket by determining if; the ticket information is valid; and the read biometric identification information comprises biometric identification information registered with the server in association with the ticket information (the “registered biometric identification”); whether there are any restrictions not met; and communicating by the server to the entrance of the event whether to grant access to the event to the ticketholder.

Determining if the ticket information is valid may comprise verifying that the ticket information matches the ticket information registered with the server for the event. Determining by the server whether to grant access to the event to a ticket holder of the ticket may further comprise determining whether a predetermined period of time has not expired since the registered biometric indemnification information was first read at a location or on a mobile device at the event away from the entrance to the venue.

The method may further comprise determining by the server if the read biometric identification information is valid, and determining by the server whether to grant access to the event to a ticketholder of the ticket and further comprise determining whether registered value and/or biometric identification information has been validated.

The method may further comprise communicating with an additional server, which may be a third party server, to see if there are any additional restrictions for entry and if so, whether those restrictions have been met and communicating to the entrance of the event whether or not to grant access to the ticketholder.

The method may further comprise communicating by the server to the mobile device or location at the event away from the entrance to the event where the read biometric identification was read whether the read biometric identification information is valid.

Determining by the server if the read biometric identification information is valid may comprise verifying that the read biometric information matches biometric information registered with the server in association with one or more tickets.

The registered biometric identification information may further comprise biometric identification from a plurality of biometric identifications. The registered biometric identification information may comprise primary biometric identification information and secondary biometric identification information, and determining by the server whether to grant access to the event to the ticketholder of the ticket may further comprise determining if the secondary biometric identification has been used to grant access to more than a predetermined number of tickets.

According to another aspect, there is provided a system for identifying the actual attendee to an event comprising: a biometric validation unit for placement at the event away from the entrance of the event, the biometric identification information validation unit configured to read or receive biometric identification information (“read biometric identification information”) from a biometric identification and communicate the read biometric information to a server; a ticket validation unit for placement at the entrance of the event, the ticket validation unit configured to read ticket information from a ticket and communicate the ticket information to the server; the server configured to: receive biometric information from the biometric identification validation unit; receive ticket information from the ticket validation unit; determine whether to grant access to the event to a ticketholder of the ticket by determining if: the ticket information is valid; and the read biometric identification information comprises biometric information registered with the server in association with the ticket information (the “registered biometric identification”) and whether there are any restrictions not met; and communicate to the ticket validation unit whether to grant access to the event to the ticketholder.

Determining if the ticket information is valid may comprise verifying that the ticket information matches the ticket information registered with the server and any subsequent servers for the event.

Determining whether to grant access to the event to a ticketholder of the ticket may further comprise determining whether a predetermined period of time has not expired since the registered biometric identification information was first read on a mobile device or at a location at the event away from the entrance to the event.

The server may be further configured to determine if the read biometric information is valid and determining whether to grant access to the event to a ticketholder of the ticket may further comprise determining if the registered biometric identification information has been validated.

The server may be further configured to communicate to the mobile device or biometric identification validation unit where the read biometric identification information was read, and the biometric identification unit and/or the mobile device may be further configured to display whether the read biometric identification information is valid.

Determining if the read biometric information is valid may comprise verifying that the read biometric identification information matches biometric identification information registered with the server in association with one or more tickets.

The registered biometric identification information may comprise biometric identification from a plurality of biometric identifications. The registered biometric identification information may comprise primary biometric identification information and secondary biometric identification information, and determining by the server whether to grant access to the event to a ticketholder of the ticket may further comprise determining if the secondary market biometric identification has been used to grant access to more than a predetermined number of tickets.

According to another aspect, there is provided a method for identifying the actual attendee at a live event comprising: receiving by a server mobile device biometric identification (“received mobile device biometric information”) information communicated from one or more mobile devices; reading ticket information from a ticket at the entrance of the event and communicating the ticket information to the server; determining by the server whether to grant access to the event to a ticketholder of the ticket by determining if: the ticket information is valid; the received mobile device information comprises mobile biometric identification information registered with the server in association with the ticket information (the “registered mobile device biometric identification information”); and whether there are any restrictions not met; and communicating by the server to the entrance of the event whether to grant access to the event to the ticketholder.

Determining if the ticket information is valid may comprise verifying that the ticket information matches ticket information registered with the server for the event.

“Received mobile device biometric information” may comprise any form of biometrics of an individual including but not limiting face ID, retina ID, finger or thumbprint ID and voice ID (as well as “palm prints” utilized, for example, by the recent “Amazon One” service).

As an added confirmation, the method may further comprise using a phone's IMEI number registered in the biometric ID server such that when the ticketholder verifies their identity biometrically, the verification is accepted only from the phone associated with the IMEI number.

Determining by the server whether to grant access to the event to a ticketholder of the ticket may further comprise determining whether a predetermined period of time has not expired since the registered mobile device biometric information was first communicated to the server by the mobile device associated with the mobile device biometric identification information.

The method may further comprise determining by the server if the received mobile device biometric identification information is valid.

The method may further comprise communicating by the server to each mobile device whether the received mobile device biometric identification information from the mobile device is valid.

Determining by the server if the received mobile biometric identification information is valid may comprise verifying that the received mobile device biometric information matched mobile device biometric identification information registered with the server in association with one or more tickets.

The method may further comprise determining by the server if biometric identification information registered with the server in respect of the ticketholder and/or ticket is valid, and determining by the server whether to grant access to the event to a ticketholder of the ticket may further comprise determining if the biometric identification information registered in association with the ticketholder and/or ticket has been validated.

The method may further comprise communicating by the server to each mobile device whether the biometric identification registered with the server in association with the ticketholder and/or ticket is valid.

The method may further comprise receiving by the server biometric identification information (“received biometric identification information”) from one or more mobile devices in respect of the ticketholder and/or ticket and determining by the server whether to grant access to the event to a ticketholder of the ticket may further comprise determining if biometric identification information registered with the server in association with the ticket and/or ticketholder has been validated.

The method may further comprise communicating by the server to each mobile device whether the received biometric identification is valid.

The method may further comprise receiving by the server positioning information (“received positioning information”) from the one or more mobile devices and determining by the server if the received positioning information is valid, and determining by the server whether to grant access to the event to a ticketholder of the ticket may further comprise determining by the server whether positioning information has been received from the mobile device associated with the registered mobile device identification information and whether the positioning information has been validated.

The method may further comprise communicating by the server to each mobile device whether the received positioning information from the mobile device is valid.

Determining by the server if the received positioning information is valid may comprise determining if the mobile device is within a predetermined minimum proximity to the event; this determination may need to be done within a predetermined time in advance of the start of the event. In another embodiment, an additional “ticket checkpoint” within this minimum proximity (but before an attendee is identified) could also serve to reduce the likelihood of fraud. In yet another embodiment, a “security perimeter” (e.g., checking for explosive devices or other weapons) could extend beyond the maximum proximity, enhancing security even before an attendee is identified. Moreover, weapons detection technology within this security perimeter could be employed to detect a weapon on a not-yet-identified attendee whose picture could be taken. Instead of utilizing current technology to track that person manually (e.g., with security personnel carrying mobile devices displaying that picture), the picture could instead be matched automatically with a database of attendee pictures, thereby providing not only the means to disable the attendee's ticket at an access control gate, but also to track the attendee before they even reach that gate based on additional account information (e.g., a mobile phone).

According to another aspect, there is provided a system for identifying an attendee at a live event comprising: a ticket validation unit for placement at the entrance of the event, the ticket validation unit configured to read ticket information from a ticket and communicate the ticket information to the server; the server configured to: receive biometric identification information (“received biometric identification information”) from one or more mobile devices; receive ticket information from the ticket validation unit; determine whether to grant access to the event to a ticketholder of the ticket by determining if: the ticket information is valid; and the biometric identification information comprises biometric identification information registered with the server in association with the ticket information (“the registered biometric identification”); whether there are any restrictions not met; and communicate to the ticket validation unit whether to grant access to the event to the ticketholder.

Determining if the ticket information is valid may comprise verifying that the ticket information matches ticket information registered with the server for the event.

Determining whether to grant access to the event to a ticketholder of the ticket may further comprise determining whether a predetermined period of time has not expired since the registered mobile device biometric identification information was first communicated to the server by the mobile device associated with the mobile device biometric identification information.

The server may be further configured to determine if the received mobile device biometric information is valid. The server may be further configured to communicate to each mobile device whether the received mobile device biometric identification information from the mobile device is valid.

Determining if the received mobile device biometric identification information is valid may comprise verifying that the received mobile device biometric information matches the mobile device biometric identification information registered with the server in association with one or more tickets.

The server may be further configured to determine if biometric identification registered with the server in association with received mobile device biometric identification information is valid, and determining whether to grant access to the event to a ticketholder of the ticket may further comprise determining if the biometric identification information registered in association with the registered mobile device biometric identification information has been validated.

The server may be further configured to communicate to each mobile device whether the biometric identification registered with the server in association with the received mobile device biometric identification information from the mobile device is valid.

The server may be further configured to receive biometric identification information (“received biometric identification information”) from one or more mobile devices in association with the received mobile device biometric identification information and determine whether the received biometric identification information is valid, and determining whether to grant access to the event to a ticketholder of the ticket may further comprise determining if biometric identification information registered with the server in association with the registered mobile device biometric identification information has been validated.

The system may further comprise communicating by the server to each mobile device whether the received biometric identification is valid.

The server may be further configured to receive positioning information (“received positioning information”) from one or more mobile devices and determine if the received positioning information is valid, and determining whether to grant access to the event to a ticket holder of the ticket may further comprise determining whether the positioning information has been received from the mobile device associated with the registered mobile device biometric identification information and whether the positioning information has been validated.

The server may be further configured to communicate to each mobile device whether the received positioning information from the mobile device is valid.

Determining if the received positioning information is valid may comprise determining if the mobile device is within a predetermined minimum proximity to the event.

According to another aspect, there is provided a method for identifying an attendee at a live event comprising: receiving at a server a request from a purchaser to purchase a ticket to the event; requesting by the server from the purchaser biometric information of the purchaser; receiving at the server biometric identification information; confirming that the biometric identification is in real time; receiving at the server some form of government photo identification or other acceptable or equivalent photo identification; matching the biometric identification with the government photo identification; registering by the server the biometric identification information in association with the ticket receiving any restrictions from additional outside servers to be associated with the ticket.

The server may be configured to ask the purchaser to download a selfie of themselves on a mobile device or desktop for facial liveness detection.

The biometric system will measure and analyze physical characteristics and reactions in order to determine if a biometric sample is being captured from a living subject who is present at the point of capture.

The server can request the purchaser to capture a photo of their passport, drivers' licence, visa, known travellers' card, or any form of government photo ID acceptable for facial comparison.

The system may match up the biometric identification information with the captured photo ID information and be registered by the server.

Once facial identification is confirmed, the system may request from the purchaser if they prefer to register another form of biometrics such as their voice or thumb/fingerprint in replacement of their selfie to be stored as their registered biometric identification information. By performing this “remote identity enrollment” (where facial identification may be practically required), a “proxy identity” (such as voice) can be substituted to avoid the need to store one's facial identity for subsequent identity confirmation or authentication. Moreover, “two-factor authentication” (“2FA”) policies can also be accommodated where desired by adding one or more of these proxy identity options.

The system may further be configured to receive at the server a request from the purchaser to update the biometric identification information of the purchaser registered in association with the ticket and registering by the server the update to the biometric identification information in association with the ticket. For example, an attendee might update vaccination status or other health criteria.

According to another aspect, there is provided a method for identifying an attendee at a live event comprising: receiving any restrictions from additional outside servers to be associated with the ticket that may include: (i) whether an attendee has been vaccinated for a specific virus; (ii) whether an attendee has had a specific virus; (iii) whether an attendee has passed an antibody test; and/or (iv) whether an attendee has tested negative for COVID-19 and transforming those restrictions into either allowing a ticket to be activated for entry via mobile device or ticket validation unit, or not allowed.

According to another aspect, there is provided a method for identifying an attendee at areas within a venue at which a live event is being held, in which entering the areas within the venue requires satisfying additional access control in addition to the access control deployed to gain admittance to the event-at-large. Example areas within the live event comprise VIP, lounge, and/or backstage areas; and stores and kiosks for food, beverage, and merchandise purchases.

According to another aspect, there is provided a method for identifying an attendee at a live event, the method permitting the attendee to leave and return to the venue (or to a specific room or area within the venue, such as a club, loge or suite level), the method comprising: recognizing via positioning that the attendee has exited the event or area and their ticket needs to be reactivated (or rechecked for a different category of access control), or receiving a manual indication via the ticket validation unit that the person has exited; requiring the attendee to check-back in for re-entry or re-validation; and in the event that the attendee turned off mobile device location services, requesting the attending via email to register they have left. In some aspects, failing to do may automatically tell the system the attendee is outside of the geofence zone and left the event or area.

According to another aspect, there is provided a non-transitory computer readable medium having program code stored thereon, wherein the program code is executable by a processor and, when executed by the processor, causes the processor to perform the method of any of the foregoing aspects and suitable combinations thereof.

According to another aspect, an attendee's identity (captured at the time of “enrollment” or ticket purchase) is confirmed or authenticated remotely (e.g., via a mobile phone or other wireless device) when the attendee is within a predefined proximity to the event. Once the attendee's identity is confirmed, the “normal” access control features (e.g., a gate ticket scanner) are activated. As a result, the attendee can utilize whatever form of ticketing is otherwise required for the event without requiring further identity confirmation. This process avoids the need to modify existing access control infrastructure, and avoids the corresponding delays and security risks that ensure when identity or health status checks are performed at the access control point (i.e., at the transition from an uncontrolled environment to a controlled environment). Moreover, it enables subsequent identity confirmation checks to be performed quickly (e.g. via use of a proxy identity, biometric or otherwise) without any change in infrastructure or additional labor.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

FIG. 1 is a system diagram of a ticket management system according to one embodiment.

FIG. 2 is a process diagram of a method of purchasing an event ticket employed by the ticket management system shown in FIG. 1.

FIG. 3 is a process diagram of a method of modifying the registered ticketholder associated with an event ticket employed by the ticket management system shown in FIG. 1.

FIG. 4 is a process diagram of a method of modifying the registered ticketholder valued identification associated with an event ticket employed by the ticket management system shown in FIG. 1.

FIG. 5 is a system diagram of a ticket authentication system according to another embodiment.

FIG. 6 is a process diagram of method of authenticating a ticket at an event employed by the ticket authentication system shown in FIG. 5.

FIG. 7 is a system diagram of a secondary market system according to another embodiment.

FIG. 8A is a process diagram of a method of posting one or more previously purchased tickets for sale employed by the secondary market system shown in FIG. 7.

FIG. 8B is a process diagram of method of purchasing one or more previously purchased tickets that have been posted for sale employed by the secondary market system shown in FIG. 7.

FIG. 9A is a process diagram of a method of bidding for the purchase of a previously purchased ticket employed by the secondary market system shown in FIG. 7.

FIG. 9B is a process diagram of a method of offering a previously purchased ticket to fulfill a bid for the purchase of a ticket employed by the secondary market system shown in FIG. 7.

FIG. 10 is a system diagram of a mobile device for use as a valued identification validation unit according to another embodiment.

FIG. 11 is a process diagram of a method of offering for transfer or resale of tickets to event segments of a segmented event.

FIG. 12 is a system diagram of a ticket authentication system according to another embodiment.

FIG. 13 is a process diagram of a method of authenticating a ticket at an event employed by the ticket authentication system shown in FIG. 12.

FIG. 14 is an example screen of a mobile application confirming an identify and health status of a ticketholder, for use in conjunction with the system of FIG. 12 and method of FIG. 13.

FIG. 15 is a process diagram of an example method of generating and storing an identification record, which can be used to verify the identity of a ticketholder, in a data server.

FIG. 16 is a system diagram depicting various devices networked to a biometric server used to monitor when an identified ticketholder is present at various monitored areas.

FIG. 17 is system diagram depicting example relationships between a particular ticketholder being contact traced by virtue of their identity and location being recorded in various different areas.

This summary does not necessarily describe the entire scope of all aspects. Other aspects, features and advantages will be apparent to those of ordinary skill in the art upon review of the following description of specific embodiments.

### DETAILED DESCRIPTION

Throughout the detailed description the term “valued identification” is used to refer to identification that is registered in the name of the owner of the identification and can be charged a value for the purchase of goods and/or services, including without limitation, credit cards, debit credit cards, prepaid credit cards, closed-loop or semi-closed loop credit cards that can only be used with specific merchants, and charge cards. Valued identification may be in the form of a card or other media that contains valued identification information, such as for example: magnetic strips; integrated circuits; computer readable mediums; contactless media using near field communications technologies, such as, for example, radio frequency identification tags, PayPass™, contactless payment stickers, mobile communication devices; or other storage media known to one skilled in the art. The valued identification information may include account number information, owner information, expiration date information, service code information, verification information (i.e. PIN or card verification information, such as, for example, card verification code (CVC), card verification valued code (CVVC), card verification value (CVV), card security code (CSC), card verification data (CVD), verification code (V Code) or card code verification (CCV)), and other similar information known to one skilled in the art.

In addition, throughout the disclosure where a server or computer is referenced it may include one or more servers or computers located at one or more locations communicating through one or more networks. Where a processor is referenced it may include one or more processors located at one more locations communicating through one or more networks, including without limitation, application specific circuits, programmable logic controllers, field programmable gate arrays, microcontrollers, microprocessors, virtual machines, electronic circuits and other processing devices known to one skilled in the art. Where a computer readable medium is referenced it may include one or more computer readable mediums located at one more locations communicating through one or more networks, including without limitation, random access memory, flash memory, read only memory, hard disc drives, optical drives and optical drive media, flash drives, and other computer readable storage media known to one skilled in the art. Where a network is referenced it may include one or more networks, including without limitation, local area networks, wide area networks, intranets, the Internet, and other networks known to one skilled in the art. Where a communication, transmission or informing of information is referenced it may be communicated over any electronic communication medium and in any format known to one skilled in the art, including without limitation, wired or wireless mediums, compressed or uncompressed formats, encrypted or unencrypted formats, email, facsimile, Short Message Service or text messages, Multimedia Messaging Service or multimedia messages, instant messaging, and website posts.

### Primary Market Ticket Management System

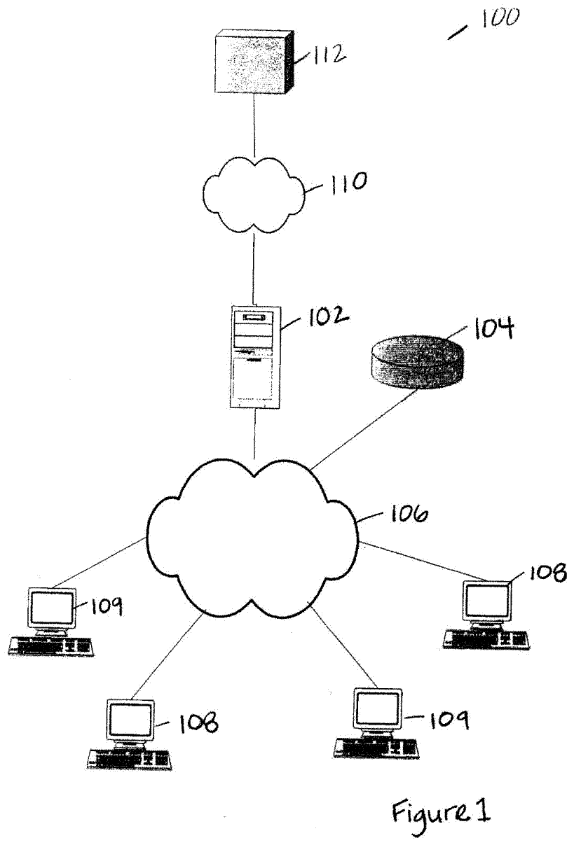

Referring to FIG. 1 a primary market ticket management system 100 is shown. The system 100 enables users to purchase tickets to an event that are made available by original tickets vendors and allocate purchased tickets to specified ticketholders. The system 100 comprises a ticket server 102, a data server 104, a network 106, user terminals 108, ticket vendors 109, a network 110, and a valued identification authorization system 112.

The ticket server 102 comprises a processor and a computer readable medium (not shown). The computer readable medium contains instructions stored therein that when executed by the processor perform the ticket management method as further described below. The ticket server 102 communicates with the data server 104, user terminals 108, and ticket vendors 109 through network 106, and the valued identification authorization system 112 through network 110.

The data server 104 comprises a processor and a computer readable medium (not shown). The computer readable medium contains instructions stored therein that when executed by the processor facilitate the management, communication, access and storage of data on the data server 104. The data may comprise information respecting the identification, validity, availability, purchase, ownership and allocation of event tickets. In one embodiment, the data consists of account information (i.e. user name, password, contact information, mobile device identification information, etc.), the status of each ticket (i.e. purchased, available for purchase, etc.), ticket information (i.e. ticket identification information, event information, seat information, price, price category, etc.), ownership information (i.e. the original purchaser of each ticket, the ticketholder of each ticket, contact information of the original purchaser, contact information of the ticketholder, mobile device identification information, etc.), purchase restrictions (i.e. maximum number of tickets that can be purchased by each purchaser, prohibited purchasers, permitted purchasers, etc.), and valued identification information of the ticketholder of each ticket. The data server 104 communicates with the ticket server 102 through network 106. In the alternative, the data server 104 may communicate with the ticket server 102 over a separate network from network 106. In the further alternative, the data server 104 may form part of the ticket server 102. It should be noted that various different types of ticket information can be accommodated, such as a bar code, QR code, RFID chip, etc. Moreover, different “ticket instruments” can be accommodated as desired (e.g., paper tickets, mobile tickets, wristbands, etc), thus enabling existing infrastructure to be utilized for access control after being enabled once an attendee's identify has been authenticated and linked to these different ticket instruments (akin to a proxy identity).

Users can access the ticket server 102 through network 106 via a registration device, such as, user terminals 108 and ticket vendors 109. User terminals 108 may be a computer, cellular phone, personal digital assistant, gaming device or other communication device capable of communicating with a server through a network as known to one skilled in the art. The ticket vendors 109 may be a person or an automated telephone system and users may communicate with the ticket vendors 109 over the phone or in person at designated locations. In one embodiment, the ticket server 102 hosts a website and users and ticket vendors communicate with the ticket server 102 through the website via a web browser running on the user terminals 108 and ticket vendors 109.

The valued identification authorization system 112 contains information pertaining to valued identification and enables the authorization, preauthorization and charging of monetary amounts to an account associated with the valued identification. The valued identification authorization system 112 communicates with the ticket server 102 through network 110.

### Primary Market Ticket Management Method

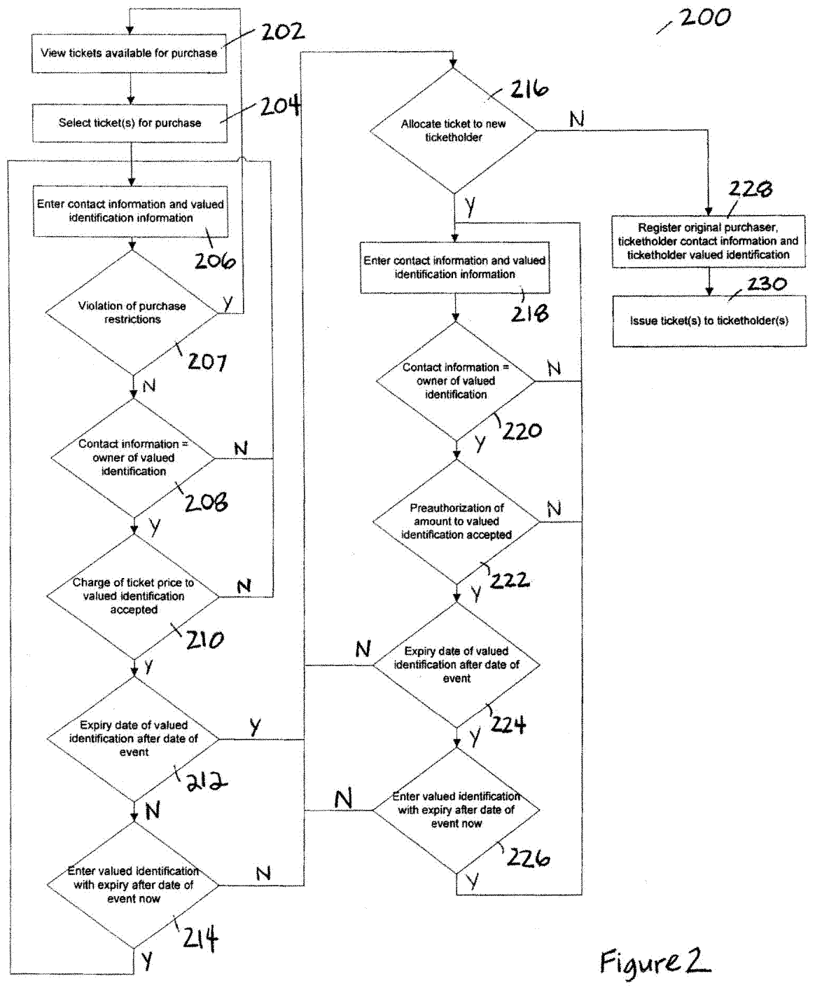

Referring to FIG. 2, a method 200 of purchasing an event ticket using system 100 is shown. In block 202, a purchaser communicates with the ticket server 102 through a user terminal 108 or ticket vendor 109 requesting a listing of tickets that are available for purchase for a particular event. The ticket server 102 queries the data server 104 to acquire the listing and communicates the listing to the user terminal 108 or ticket vendor 109. In block 204, the purchaser selects one or more tickets for purchase. The ticket server 102 then holds the selected tickets for a predetermined period during which other purchasers are prohibited from selecting the held tickets for purchase.

In block 206, the ticket server 102 prompts the purchaser to enter the purchaser's account information previously registered with the ticket server 102 or create a new account with the ticket server 102. If the purchaser elects to create a new account, the ticket server 102 prompts the purchaser to enter desired account information, the purchaser's contact information and the purchaser's valued identification information (including biometric identification info, health status info, etc.). The ticket server 102 then communicates with the data server 104 to register the new account information in association with the purchaser. If the purchaser enters previously registered account information, the ticket server 102 queries the data server 104 to authenticate the account information. If the account information is invalid, the ticket server 102 prompts the purchaser for valid account information or the creation of a new account, otherwise, the ticket server 102 queries the data server 104 for the purchaser's contact information and valued identification information registered with the purchaser's account and provides this information to the purchaser. The ticket server 102 then prompts the purchaser to proceed with the registered contact information and valued identification information or enter new contact information and/or valued identification information. If the purchaser enters new contact information and/or valued identification information, the ticket server 102 communicates and registers this information with the data server 104.

The account information may comprise a user name, password, valued identification information, contact information, mobile device identification information, or other authentication information known to one skilled in the art. In one embodiment the account information consists of a user name and password. The purchaser's contact information may comprise name, home address, home phone number, cellular phone number, email address, and other forms of contact information known to one skilled in the art. In one embodiment, the purchaser's contact information consists of the purchaser's name, address, and email address. The purchaser's valued identification information may comprise account number information, owner information, expiration date information, service code information, verification information (i.e. PIN or card verification information, such as, for example, card verification code (CVC), card verification valued code (CVVC), card verification value (CVV), card security code (CSC), card verification data (CVD), verification code (V Code) or card code verification (CCV)), and other similar information known to one skilled in the art. In one embodiment, the purchaser's valued identification information consists of account number information, owner information, and expiration date information. In at least some embodiments where a credit card is used as valued identification, all or part of the credit card number is obtained as the account number information. For example, instead of obtaining a full 16 digit credit card number, the ticket server 102 obtains only the first 6 and last 4 digits of the credit card number and stores a hash of those numbers for future reference in the data server 104. This increases cybersecurity as only a 1) hash of 2) part of the entire credit card number is stored by the system 100.

In block 207, the ticket server 102 queries the data server 104 to determine if any purchase restrictions have been violated by the purchaser. Purchase restrictions may comprise maximum number of tickets that can be purchased by each purchaser, prohibited purchasers, permitted purchasers, and other restrictions known to one skilled in the art. In one embodiment the purchase restrictions consist of the maximum number of tickets that can be purchased by each purchaser. If the purchaser has violated a purchase restriction, the ticket server 102 proceeds to block 202, otherwise, the ticket server 102 proceeds to block 208. In block 208, the ticket server 102 compares the purchaser's contact information to the ownership information of the purchaser's valued identification. If the purchaser's contact information does not match the ownership information of the purchaser's valued identification the ticket server 102 informs the purchaser and proceeds to block 206, otherwise, the ticket server 102 proceeds to block 210. In block 210, the ticket server 102 communicates the purchaser's valued identification information to the valued identification authorization system 112 through network 110 along with a request to charge the price of the tickets to the account associated with the purchaser's valued identification. If the charge request is denied by the valued identification authorization system 112, the ticket server 102 informs the purchaser and proceeds to block 206, otherwise, the ticket server 102 proceeds to block 212.

In block 212, the ticket server 102 compares the expiry date of the purchaser's valued identification (or health status info, such as an expiration date of a vaccination) to the date of the event. If the expiry date of the purchaser's valued identification is after the date of the event, the ticket server 102 proceeds to block 216, otherwise, the ticket server 102 proceeds to block 214. In block 214, the ticket server 102 informs the purchaser that prior to the date of the event the purchaser must register valued identification with the ticket server 102 having an expiry date after the date of the event. The purchaser is prompted to either enter new valued identification having an expiry date after the date of the event or indicate that the purchaser will enter such valued identification at a later time prior to the date of the event (i.e. by selection of a check box or other manner of indication known to one skilled in the art). If the purchaser selects the entry of new valued identification, the ticket server 102 proceeds to block 206, otherwise, the ticket server 102 proceeds to block 216.

In block 216, the ticket server 102 prompts the purchaser to select one or more of the purchased tickets for which the purchaser desires to allocate to a new ticketholder other than the purchaser. If the purchaser desires to allocate any of the purchase tickets to a new ticketholder, the ticket server 102 proceeds to block 218, otherwise the ticket server 102 proceeds to block 228. In block 218, the ticket server 102 prompts the purchaser to select a ticket and enter the contact information and valued identification information of the new ticketholder. The ticketholder's contact information may comprise name, age, home address, home phone number, cellular phone number, email address, and other forms of contact information known to one skilled in the art. In one embodiment, the ticketholder's contact information consists of the ticketholder's name, address, and email address. The ticketholder's valued identification information may comprise account number information, owner information, expiration date information, service code information, verification information (i.e. PIN or card verification information, such as, for example, card verification code (CVC), card verification valued code (CVVC), card verification value (CVV), card security code (CSC), card verification data (CVD), verification code (V Code) or card code verification (CCV)), and other similar information known to one skilled in the art. In one embodiment, the ticketholder's valued identification information consists of account number information, owner information, and expiration date information.

In block 220, the ticket server 102 compares the new ticketholder's contact information to the ownership information of the new ticketholder's valued identification. If the ticketholder's contact information does not match the ownership information of the ticketholder's valued identification the ticket server 102 informs the purchaser and proceeds to block 218, otherwise, the ticket server 102 proceeds to block 222. In block 222, the ticket server 102 communicates the ticketholder's valued identification information to the valued identification authorization system 112 through network 110 along with a request for a preauthorization of a predetermined amount. In one embodiment, the predetermined amount is the price of the ticket. If the preauthorization request is denied by the valued identification authorization system 112, the ticket server 102 informs the purchaser and proceeds to block 218, otherwise, the ticket server 102 proceeds to block 224.

In block 224, the ticket server 102 compares the expiry date of the new ticketholder's valued identification to the date of the event. If the expiry date of the new ticketholder's valued identification is after the date of the event, the ticket server 102 proceeds to block 216, otherwise, the ticket server 102 proceeds to block 226. In block 226, the ticket server 102 informs the purchaser that prior to the date of the event the purchaser or ticketholder must register valid ticketholder valued identification information with the ticket server 102 having an expiry date after the date of the event. The purchaser is prompted to either enter new ticketholder valued identification information having an expiry date after the date of the event or indicate that the purchaser and/or ticketholder will enter new ticketholder valued identification at a later time prior to the date of the event (i.e. by selection of a check box or other manner of indication known to one skilled in the art). If the purchaser selects the entry of new ticketholder valued identification information, the ticket server 102 proceeds to block 218, otherwise, the ticket server 102 proceeds to block 216.

In block 228, the ticket server 102 instructs the data server 104 to update the information associated with the purchased tickets. Specifically, for each purchased ticket the ticket server 102: (a) sets the status of the ticket as “purchased”; (b) registers the purchaser as the original purchaser of the ticket; (c) registers the purchaser's contact information and valued identification information; and (d) registers the ticketholder's contact information and valued identification information (including, for example, biometric identification info, health status info, etc).

In block 230, the ticket server 102 issues the purchased tickets to the registered ticketholder of each ticket. In one embodiment the ticket is an electronic ticket that is delivered to the registered ticketholder via email, text message, the website hosted by the ticket server 102, or other electronic communication method known to one skilled in the art. In an alternative embodiment, the ticket may be a physical ticket that is sent to the registered ticketholder via mail or courier, or picked up by the ticketholder at a specified location.

In a further alternative embodiment, electronic tickets allocated to ticketholder's other than the registered original purchaser may be encrypted such that the ticket cannot be easily transferred to a third party. For example, in block 230, the ticket server 102 issues an email message to the registered ticketholder of each ticket. The message instructs the ticketholder to access the web page hosted by the ticket server 102. When the ticketholder accesses the web page, the ticket server 102 prompts the ticketholder for the ticketholder's registered contact information and valued identification information. In one embodiment, the ticketholder is prompted for the ticketholder's email address and the last 5 digits of the account number of the ticketholder's valued identification information. The ticket server 102 then queries the data server 104 to validate the contact information and valued identification information entered by the ticketholder. If the information is valid, the ticket server 102 displays an electronic ticket on the webpage in a form that can only be printed and not saved or forwarded, in a manner is known to one skilled in the art. The ticketholder can print a copy of the ticket to take to the event, but cannot easily electronically transfer the ticket to a third party. Optionally, the ticket server 102 may store the Internet Protocol address of the user terminal 108 that the ticketholder is using to access the ticket server 102. If the ticketholder or a third party subsequently attempts to access the ticket server 102 from user terminal 108 having a different Internet Protocol address than the user terminal 108 first used to access the ticket serve 102, the ticket server 102 may deny the user access to the electronic ticket or prompt the user for additional validation information.

At any time up to an including the date of the event, the registered original purchaser of a ticket may access the ticket server 102 to reissue the ticket, reallocate the ticket, change the registered ticketholder contact information, or change the registered ticketholder valued identification. In addition, at any time up to an including the date of the event, the registered ticketholder may access the ticket server 102 to change the registered ticketholder valued identification if the registered ticketholder valued identification will expire prior to the date of the event.

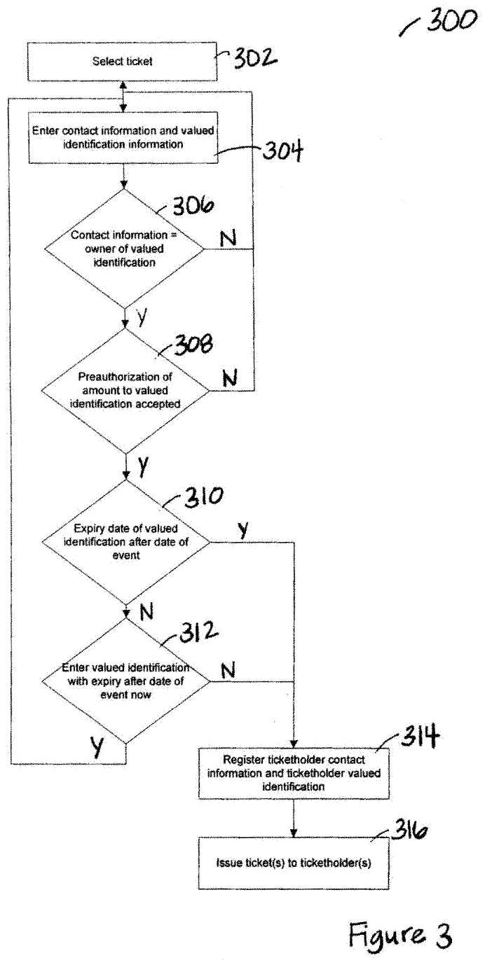

Referring to FIG. 3, a method 300 of reallocating a ticket using system 100 is shown. In block 302, the registered original purchaser selects a ticket to which the purchaser desires to change the registered ticketholder contact information. In block 304, the ticket server 102 prompts the registered original purchaser to enter the new ticketholder's contact information and valued identification information, in the same form as described above in block 218 of method 200. In block 306, the ticket server 102 compares the new ticketholder's contact information to the ownership information of the new ticketholder's valued identification. If the ticketholder's contact information does not match the ownership information of the ticketholder's valued identification the ticket server 102 informs the purchaser and proceeds to block 304, otherwise, the ticket server 102 proceeds to block 308. In block 308, the ticket server 102 communicates the ticketholder's valued identification information to the valued identification authorization system 112 through network 110 along with a request for a preauthorization of a predetermined amount. In one embodiment, the predetermined amount is the price of the ticket. If the preauthorization request is denied by the valued identification authorization system 112, the ticket server 102 informs the registered original purchaser and proceeds to block 304, otherwise, the ticket server 102 proceeds to block 310.

In block 310, the ticket server 102 compares the expiry date of the new ticketholder's valued identification to the date of the event. If the expiry date of the new ticketholder's valued identification is after the date of the event, the ticket server 102 proceeds to block 314, otherwise, the ticket server 102 proceeds to block 312. In block 312, the ticket server 102 informs the registered original purchaser that prior to the date of the event the registered original purchaser or ticketholder must register valid ticketholder valued identification information with the ticket server 102 having an expiry date after the date of the event. The registered original purchaser is prompted to either enter new ticketholder valued identification information having an expiry date after the date of the event or indicate that the purchaser and/or ticketholder will enter new ticketholder valued identification at a later time prior to the date of the event (i.e. by selection of a check box or other manner of indication known to one skilled in the art). If the purchaser selects the entry of new ticketholder valued identification information, the ticket server 102 proceeds to block 304, otherwise, the ticket server 102 proceeds to block 314.

In block 314, the ticket server 102 instructs the data server 104 to register the new ticketholder's contact information and valued identification information in association with the ticket. In block 316, the ticket server 102 reissues the ticket to the new ticketholder. In one embodiment the ticket is an electronic ticket that is delivered to the registered ticketholder via email, text message, the website hosted by the ticket server 102, or other electronic communication method known to one skilled in the art. Optionally, electronic tickets may be encrypted, as described above in relation to method 200, such that the ticket cannot be transferred to a third party. In the alternative, the ticket may be a physical ticket that is sent to the registered ticketholder via mail or courier, or picked up by the ticketholder at a specified location.

In a further alternative embodiment, in block 230 of method 200 and block 316 of method 300, the registered original purchaser is prompted to select one of the following methods of delivery for the purchased tickets: 1) delivery of the physical tickets by courier or mail; 2) pickup of physical tickets at location, for example, at a will call window; or 3) electronic delivery of electronic tickets. Optionally, the purchaser may select a different method of delivery for each ticket. Where electronic delivery is selected, the ticket server 102 will delay electronic delivery of the electronic tickets to the registered ticketholders until a predetermined period before the start of the event, such as, for example, 24 hours prior to the start of the event. In the alternative, the predetermined period may be configured to be any period prior to the start of the event. In the further alternative, the electronic delivery of electronic tickets may be delayed until the valued identification associated with each ticket is subsequently verified at a predetermined minimum period prior to the start of the event as further described below. Amongst other benefits, the delayed delivery of electronic tickets to the predetermined period places an additional impediment to ticketholders attempting to resell their electronic tickets using unauthorized channels.

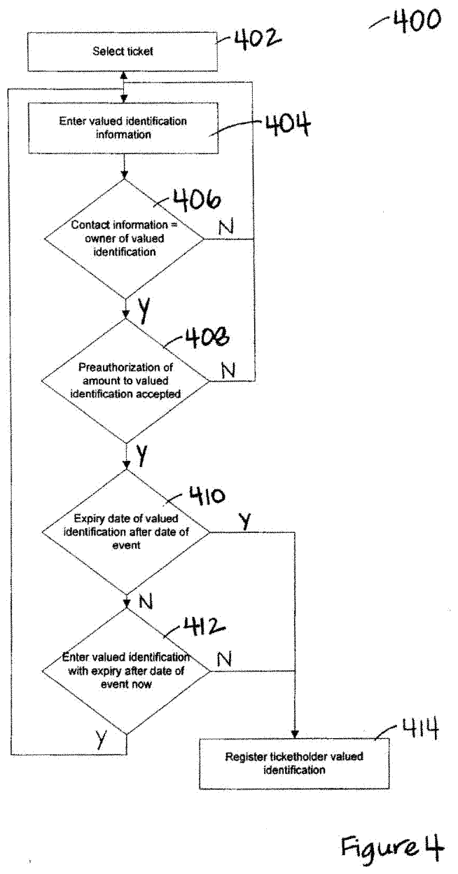

Referring to FIG. 4, a method 400 of changing the registered ticketholder valued identification information using system 100 is shown. In block 402, the registered original purchaser or registered ticketholder selects a ticket for which the registered ticketholder valued identification information is desired to be changed. In block 404, the ticket server 102 prompts the registered original purchaser or registered ticketholder to enter the new ticketholder valued identification information, in the same form as described above in block 218 of method 200. In block 406, the ticket server 102 compares the registered ticketholder contact information to the ownership information of the new ticketholder valued identification. If the registered ticketholder contact information does not match the ownership information of the new ticketholder valued identification the ticket server 102 informs the registered original purchaser or registered ticketholder and proceeds to block 404, otherwise, the ticket server 102 proceeds to block 408. In block 408, the ticket server 102 communicates the new ticketholder valued identification information to the valued identification authorization system 112 through network 110 along with a request for a preauthorization of a predetermined amount. In one embodiment, the predetermined amount is the price of the ticket. If the preauthorization request is denied by the valued identification authorization system 112, the ticket server 102 informs the registered original purchaser or registered ticketholder and proceeds to block 404, otherwise, the ticket server 102 proceeds to block 310.

In block 410, the ticket server 102 compares the expiry date of the new ticketholder valued identification to the date of the event. If the expiry date of the new ticketholder valued identification is after the date of the event, the ticket server 102 proceeds to block 414, otherwise, the ticket server 102 proceeds to block 412. In block 412, the ticket server 102 informs the registered original purchaser or registered ticketholder that prior to the date of the event the registered original purchaser or ticketholder must register valid ticketholder valued identification information with the ticket server 102 having an expiry date after the date of the event. The registered original purchaser or registered ticketholder is prompted to either enter new ticketholder valued identification information having an expiry date after the date of the event or indicate that the registered original purchaser or registered ticketholder will enter new ticketholder valued identification at a later time prior to the date of the event (i.e. by selection of a check box or other manner of indication known to one skilled in the art). If the registered original purchaser or registered ticketholder selects the entry of new ticketholder valued identification information, the ticket server 102 proceeds to block 404, otherwise, the ticket server 102 proceeds to block 414. In block 414, the ticket server 102 instructs the data server 104 to register the new ticketholder valued identification information in association with the ticket.

The ticket server 102 may perform one or more verifications of the registered ticketholder valued identification information prior to and/or including the date of the event. For each verification, the ticket server 102 communicates the registered ticketholder valued identification information to the valued identification authorization system 112 through network 110 along with a request for a preauthorization of a predetermined amount. In one embodiment the predetermined amount is the price of the ticket. If the registered ticketholder contact information does not match the owner information associated with the registered ticketholder valued identification or the preauthorization request is denied by the valued identification authorization system 112, the ticket server 102 marks the registered ticketholder valued identification information associated with the ticket as invalid. Optionally, the ticket server 102 may send one or more communications to the registered original purchaser and/or the registered ticketholder informing the recipients that the registered ticketholder valued identification information is invalid and the ticketholder will not be admitted to the event until valid ticketholder valued identification information is registered with the ticket server 102. In one embodiment, the verification of the registered ticketholder valued identification is performed by the ticket server 102 on the date of the event immediately prior to the opening of the event. In an alternative embodiment, the verification of the registered ticketholder valued identification is performed by the ticket server 102 within a twenty four hour period before the start of the event.

The ticket server 102 may perform one or more checks of the expiry date of the registered ticketholder valued identification up to and/or including the date of the event. If the expiry date of the registered ticketholder valued identification is on or before the date of the event, the ticket server 102 sends one or more communications to the registered ticketholder and/or registered original purchaser informing the recipients that prior to the date of the event the registered original purchaser and/or registered ticketholder must provide the ticket server 102 with valued identification of the registered ticketholder having an expiry date after the date of the event. The ticket server 102 may communicate the verification and/or comparison to the registered ticketholder and/or registered original purchaser via email, text message, mail, courier, telephone, the web site hosted by the ticket server 102, or other communication method known to one skilled in the art.

The ticket server 102 may also send one or more communications to the registered original purchaser and/or registered ticketholder up to an including the date of the event, reminding the registered original purchaser and/or registered ticketholder that the registered ticketholder must present the registered ticketholder valued identification at the event in order to be admitted to the event.

At the conclusion of an event, the ticket server 102 may optionally instruct the data server 104 to erase all information respecting the allocation of tickets to ticketholders by the registered original purchaser. In the alternative, this information may be archived by the data server 104 for future reference.

In an alternative embodiment, method 200 and method 300 may facilitate the electronic communication of ticketholder contact information and valued identification information from a new ticketholder in order to reallocate a ticket to the new ticketholder. In blocks 218 and 304, the ticket server 102 provides the registered original purchaser with the option to: enter one or more electronic addresses for the new ticketholder, such as, for example, email address, mobile phone number, Instant Messaging address, mobile Personal Identification Number (PIN), or other electronic addresses known to one skilled in the art; or select the ticketholder from a list of pre-registered ticketholders with the ticket server 102 or data server 104. The ticket server 102 then transmits a message to the electronic address informing the new ticketholder that the registered original purchaser desires to allocate a ticket to the ticketholder.

The ticketholder may indicate their acceptance of the ticket by providing their contact information and valued identification information to the ticket server 102 by any electronic communication means known to one skilled in the art, such as, for example, through a website hosted by the ticket server 102 or another server for which a link is provided in the message, or through a software application running on the ticketholder's computing device that communicates the information to the ticket server 102. Alternatively, if the ticketholder has already registered their contact information and valued identification information with the ticket server 102 or data server 104, the ticketholder may authenticate themselves by providing their authentication information to the ticket server 102, such as, for example, user name and password, or other authentication information apparent to one skilled in the art. The ticketholder's communications with the ticket server 102 may be encrypted in order to provide additional security.

The new ticketholder's contact information and valued identification information is then validated in the manner described in blocks 220 to 224 of method 200 or blocks 306 to 312 of method 300. If the information fails validation, the ticket server 102 transmits a message to the ticketholder's electronic address providing the ticketholder with details of the failure and requesting the ticketholder to provide valid contact information and/or valued identification information. Once the new ticketholder's information is validated, the ticket server 102 informs the registered original purchaser that the ticketholder has accepted the ticket and requests the registered original purchaser to validate the information provided by the ticketholder and confirm the allocation of the ticket to the ticketholder. If the registered original purchaser confirms the allocation of the ticket to the ticketholder, the ticket server 102 registers the new ticketholder's information in association with the ticket and issues the ticket to the new ticketholder in the manner described in blocks 228 to 230 of method 200 or blocks 314 to 316 of method 300 in the embodiments described above. In the alternative, the ticket server 102 may automatically register the information associated with the ticket upon receipt of valid contact information and valued identification information from the ticketholder without requiring confirmation from the registered original purchaser.

Once the ticketholder's contact information and valued identification information is registered with the ticket server 102, the original registered purchaser may access the ticket server 102 at any time to view both the contact information and valued identification information. If the valued identification information is stored in an encrypted format, the registered original purchaser may instruct the ticket server 102 to decrypt valued identification information and present the decrypted information to the registered original purchaser. In the alternative, the original registered purchaser may be permitted to view the valued identification information of the ticketholder solely during the confirmation of the allocation of the ticket to the ticketholder such that after confirmation the original registered purchaser will no longer be able to access the ticketholder's valued identification information.

In a further alternative embodiment, the method of electronically communicating ticketholder information and valued identification information described above may be varied such that the ticketholder initiates communication with the ticket server 102. The ticketholder communicates with the ticker server 102 and identifies the registered original purchaser of a ticket for which the ticketholder desires to receive a ticket. In the same manner as described above, the ticketholder provides their contact information and valued identification information, or authenticates themselves with the ticket server 102 if they have a previously registered this information with the ticket server 102 or data server 104. The ticketholder's contact information and valued identification information is then validated as described above. Once the ticketholder's information is validated, the ticket server 102 informs the registered original purchaser that the ticketholder has provided valid contact information and valued identification to the ticket server 102. In one embodiment, the ticketholder's contact information and valued identification information is communicated to the registered original purchaser. In an alternative embodiment, only the ticketholder's contact information is communicated to the registered original purchaser. The registered original ticket purchaser may then select one or more tickets to allocate to the ticketholder. Once the registered original purchaser has made their selection, the ticket server 102 registers the ticketholder contact information and valued identification information with the ticket(s) and informs the ticketholder as to the details of the ticket(s) that have been allocated to them.

In a further alternative embodiment, the ticket server 102 may permit the registered original purchaser to register primary valued identification information and secondary valued identification information in association with a group of tickets. The registered original purchaser may specify a maximum number of the group of tickets for which the secondary valued identification information may permit access to the event. In the alternative, the maximum number of tickets permitted to be associated with the secondary valued identification information is predetermined by the ticket server 102. As further described in the ticket authentication method, when the secondary valued identification information is authenticated at the event, up to the maximum number of tickets associated with the secondary valued identification information may be permitted to enter the event. The primary valued identification may then be subsequently authenticated to permit any remaining tickets of the group of tickets (which exceed the maximum number of tickets associated with the secondary valued identification information or have not been utilized to enter the event based on the authentication of the secondary valued identification information) to enter the event. In this manner, a purchaser can control the enablement of existing access control infrastructure with respect to a group of associated tickets (e.g., a parent associated with their minor children having the same last name). For example, upon being within a predetermined proximity to an event, a parent could drop off underage children whose ticket instruments would then be accepted once they reach the gate or other access control mechanism—without requiring any additional biometric identification. In other embodiments, the ticket instruments could be categorized (children, seniors, etc.), and a notification (e.g., a different color per category) could be provided at access control, to enhance security and fraud prevention. By remotely authenticating or confirming identity (for a single attendee or a group) once within proximity to an event, but before reaching a gate or other form of access control, the present invention avoids many of the additional checks (age, health status, etc) that would otherwise require additional labor and modification of existing access control infrastructure, as well as additional security concerns due to delays and crowds within an uncontrolled environment.

In a further alternative embodiment, the original ticket vendor may restrict which registered original purchasers may add secondary valued identification information to a group of tickets. For example, the original ticket vendor may restrict this feature to season ticket holders to permit a first portion of the members of the season ticketholder's family or group to enter the event while the remaining members park the car (i.e. the season ticketholder's spouse and children may enter the event using the secondary valued identification while the season ticketholder subsequently uses the primary valued identification to enter the event).

In a further alternative embodiment, the ticket server 102 is configured to present purchasers with the view that the purchaser can expect to see from the seat that the purchaser is considering purchasing. The data server 104 stores a plurality of graphical images (each a “seat view”) depicting views at the event and registers the seat views with the associated ticket for the event. In block 202, the ticket server 102 queries the data server 104 to acquire a listing of tickets available for purchase. The ticket server 102 communicates the listing to the user terminal 108 or ticket vendor 109 along with a seat view, or link to a seat view, for each ticket in the list having an associated seat view stored in the data server 104. Before purchasing a ticket, the purchaser may select the seat view associated with the ticket to get a sense of the view from the seat or area for which the ticket relates. In one embodiment, the same seat view may be used for a predefined group of tickets, for example, the tickets relating to a block of seats at an event could have the same associated seat view representative of the view from the center seat of the block of seats. In the alternative, the seat view representative of the view from any location within the block of seats may be associated with all of the tickets in the block of seats. In the further alternative, a distinct seat view may be association with each ticket. In the further alternative, prior to purchasing a ticket, the purchaser may communicate the associated seat view to a third party via email, text message, the website hosted by the ticket server 102, or other electronic communication method known to one skilled in the art.

### Ticket Authentication System

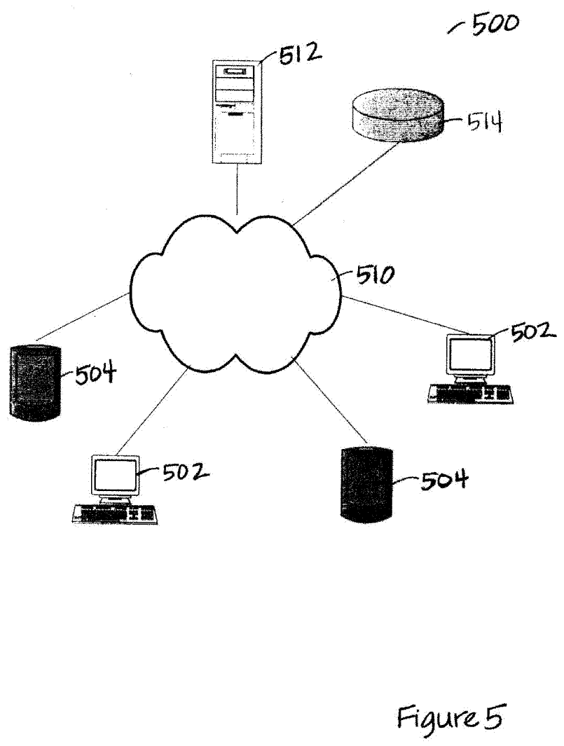

Referring to FIG. 5 a ticket authentication system 500 is shown. The system 500 authenticates event tickets presented at an event and controls access to the event by requiring event attendants to provide both an event ticket and the registered valued identification information associated with the ticket. The system 500 comprises valued identification validation units 502, ticket validation units 504, a network 510, an authentication server 512, a data server 514, and authentication assistance units (not shown).

The valued identification validation units 502 each comprise a processor, a computer readable medium, a validation indicator, and a valued identification reader (not shown). The computer readable medium contains instructions stored therein that when executed by the processor facilitates the reading of valued identification information from a piece of valued identification that is presented to the valued identification reader. The unit 502 is in communication with authentication server 512 through network 510. The unit 502 can transmit read valued identification information to the authentication server 512 and receive validation information from the authentication server 512. The validation indicator presents received validation information to the user of the unit 502. In one embodiment, the unit 502 is an automated kiosk and the validation indicator is a display. For example, the unit 502 may be a Motorola MK 2220 with a magnetic stripe reader and near field communications transceiver for reading contactless media containing valued identification information. In the alternative, the unit 502 may be any automated or non-automated device capable of providing the functionality of the unit 502 described herein. The unit 502 may be manned by an attendant or unmanned; for example, the unit 502 may be located in a box office with an attendant to assist a ticketholder with identity verification. In the further alternative, the validation indicator may comprise one or more displays, speakers, lights or other devices capable of presenting visual and/or audible information. In the yet further alternative, the valued identification validation unit 502 may comprise a display and/or a printer for presenting advertisements to users of the unit 502. Mobile phones and tablets could be employed as well in other embodiments.

The ticket validation units 504 (e.g., access control gate scanners) each comprise a processor, a computer readable medium, a ticket reader, and a validation indicator (not shown). The computer readable medium contains instructions stored therein that when executed by the processor facilitates the reading of ticket information from an event ticket that is presented to the ticket reader. The unit 504 is in communication with authentication server 512 through network 510. The unit 504 can transmit read ticket information to the authentication server 512 and receive validation information from the authentication server 512. The validation indicator presents received validation information to the user of the unit 504. The validation information may comprise an indication as whether a ticket is valid, reasons why the ticket is invalid (i.e. not a valid ticket identifier, valued identification has not bee presented to a valued identification validation unit, a predetermined period of time from the first presentation of the valued identification to the valued identification unit has expired and/or the extent to which it has expired, etc), instructions on how to handle the invalid ticket and/or other information pertaining to the validity of a ticket and valued identification known to one skilled in the art.

In one embodiment, the unit 504 is handheld wireless device (e.g. a mobile phone or tablet) operated by an event associate, the validation indicator is a display integrated in the device, and the ticket reader is a barcode reader. For example, the unit 504 may be a Motorola MC55 Enterprise Digital Assistant with a bar code reader. In the alternative, the unit 504 may be any automated or non-automated device capable of providing the functionality of the unit 504 described herein. As additional examples, the unit 504 may be an unmanned scanner (e.g., a turnstile gate with a barcode of RFID reader to respectively read a barcode or RFID on a ticket). In the further alternative, the validation indicator may comprise one or more displays, speaker, lights or other devices capable of communicating visual or audible information.

The authentication server 512 comprises a processor and a computer readable medium (not shown). The computer readable medium contains instructions stored therein that when executed by the processor perform the ticket authentication method as further described below. The authentication server 512 communicates with the valued identification validation unit 502, the ticket validation unit 504 and the data server 514 through network 510.

The data server 514 comprises a processor and a computer readable medium (not shown). The computer readable medium contains instructions stored therein that when executed by the processor facilitate the management, communication, access and storage of data on the data server 514. The data may comprises information respecting the identification, validity, ownership and allocation of event tickets. In one embodiment, the data consists of the status of each ticket (i.e. not read, read, time that the ticket was read, etc.), ticket information (i.e. ticket identification information, event information, seat information, price, price category, etc.), ownership information (i.e. the original purchaser of each ticket, the ticketholder of each ticket, contact information of the original purchaser, etc.), valued identification information of the ticketholder of each ticket and the status of the valued identification (i.e. valid, invalid, not read, read, time that the valued identification was read, etc). The data server 514 communicates with the authentication server 512 through network 510. In the alternative, the data server 514 may communicate with the authentication server 512 over a separate network from network 510. In the further alternative, the data server 514 may form part of the authentication server 512.

In one embodiment, the data server 514 is the same server as data server 104 of system 100. In an alternative embodiment, the data server 104 and data server 514 are separate servers that communicate over a network, and data stored on the data server 104 is copied to the data server 514 at a predetermined frequency.

The authentication assistance units (not shown) comprise a processor, a computer readable medium, a validation indicator, a ticket reader, a valued identification reader, and a printer. The computer readable medium contains instructions stored therein that when executed by the processor facilitates: the reading of valued identification information from a piece of valued identification that is presented to the valued identification reader; the reading of ticket information from an event ticket that is presented to the ticket reader; and the printing of tickets. Each authentication assistance unit is in communication with authentication server 512 through network 510. The authentication assistance units can transmit read valued identification information and ticket information to the authentication server 512 and receive validation information from the authentication server 512. The validation indicator presents received validation information to the user of the authentication assistance unit. In operation, the authentication assistance units may be located at a event assistance wicket or adjacent to the ticket validation unit 504, and operated by event associates to assist ticketholders with problems using the ticket authentication system 500. The authentication assistance units may also print off new tickets if instructed by an operator. In a preferred alternative embodiment, the authentication assistance unit is a handheld wireless device, such as, for example, a Motorola MC55 Enterprise Digital Assistant with a bar code reader, magstripe reader, and near field communications transceiver.

In operation, the ticket validation unit 504 is located at the entrance to the event and the valued identification validation unit 502 is located at the event away from the entrance to the event. One or more ticket validation units 504 may be located at each entrance to the event and one or more valued identification validation units 502 may be located at the event. As further described below, this separation of the ticket validation unit 504 and the valued identification unit 502 facilitates the efficient validation of event tickets and entry into the event with minimum delay. In one embodiment, there is at least one valued identification validation unit 502 for every ticket validation unit 504 present at an event and preferably 1.5 valued identification validation units 502 for every ticket validation unit 504 present at an event.

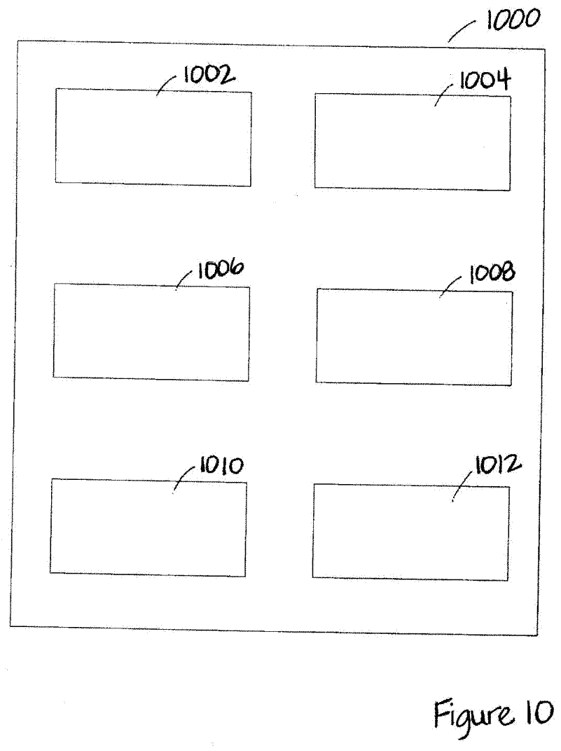

In an alternative embodiment, a ticketholder's mobile device may serve as a valued identification validation unit 502. Referring to FIG. 10, a mobile device 1000 is shown and generally comprises a display 1002 for presenting visual information to the ticketholder, an input device 1004 for receiving input from the ticketholder, a memory 1006, a processor 1008, a communication unit 1010, and a positioning unit 1012.

The memory 1006 contains statements and instructions stored therein that when executed by the processor 1008 facilitates the authentication of the ticketholder's valued identification information as further described below. The positioning unit 1012 determines the location of the mobile device 1000 using one or more Global Positioning System (GPS) receivers, cellular transceivers to triangulate the position of the mobile device between cell tower, Bluetooth transceivers to identify proximity of the mobile device 1000 to a Bluetooth device having a known location, and other methods of determining the location of a mobile device of known in the art. The communication unit 1010 communicates with the authentication server 512 to transmit one or more of authentication information, valued identification information or positioning information to the authentication server 512 and receive validation information from the authentication server 512. In the present embodiment, the mobile device 1000 is a personal digital assistant (such as, for example, a Blackberry™ smart phone having a GPS receiver and cellular transceiver), the positioning unit 1012 is a GPS receiver, and the communication unit 1010 is a cellular transceiver. In the alternative, the mobile device 1000 may be any mobile device (such as an iPhone or Android-based phone) capable of communicating with the authentication server 512 and determining its location known to one skilled in the art. In the further alternative, the positioning unit 1012 may be any technology known in the art to identify the location of a device. In the further alternative, the positioning unit 1012 and communication unit 1010 may be the same unit. In the further alternative, the mobile device 1000 may not comprises a positioning unit 1012.

In a further alternative embodiment, the original ticket vendor may exclude certain categories of tickets from the authentication process, such as, for example, tickets to corporate boxes and other areas of an event. Such tickets will not require validation of valued identification or valued identification information in order to permit the ticketholder entry to an event.

### Ticket Authentication Method

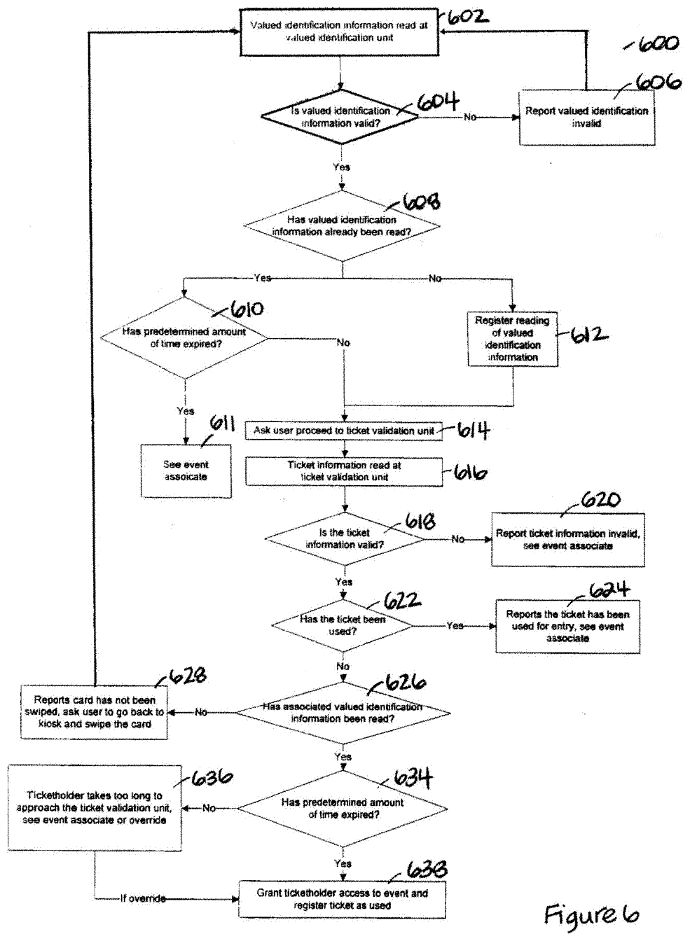

Referring to FIG. 6, a method 600 of authenticating a ticket at an event using system 500 is shown. In block 602, a ticketholder presents valued identification to the valued identification validation unit 502. The unit 502 reads valued identification information from the valued identification and transmits the information to the authentication server 512. The valued identification information may comprise account number information, owner information, expiration date information, service code information, verification information (i.e. PIN or card verification information, such as, for example, card verification code (CVC), card verification valued code (CVVC), card verification value (CVV), card security code (CSC), card verification data (CVD), verification code (V Code) or card code verification (CCV)), and other similar information known to one skilled in the art. In one embodiment, the valued identification information consists of account number information, owner information, and expiration date information. In at least some example embodiments in which the valued identification is a credit card, the valued identification validation unit 502 is configured to only read the first 6 digits and last 4 digits of a 16 digit credit card number.

In block 604, the authentication server 512 queries the data server 514 to match the read valued identification information to registered valued identification information stored in the data server 514. If the read valued identification information does not match any registered valued identification information stored in the data server 514, the method 600 proceeds to block 606. In block 606, the authentication server 512 informs the valued identification validation unit 502, and the valued identification unit 502 informs the ticketholder through the validation indicator, that the read valued identification is not valid. The method 600 then proceeds to block 602. In the example embodiments in which the unit 502 reads only the first 6 and last 4 digits of a credit card number, the unit 502 hashes these ten digits and compares this hash to the corresponding hash retrieved from the data server 514. If these two hashes match, then the read valued identification is determined to be valid; if the hashes do not match, the read valued identification is determined to be invalid. In other embodiments, this process could implement 2FA by matching multiple forms of identification, including one or more proxy identities. These factors could include facial features, voice, fingerprints, palm prints and other forms of biometric identification, as well as non-biometric factors, such as paper tickets, mobile tickets and wristbands (based on the policies deemed appropriate and implemented for any given event).

If in block 604 the read valued identification information matches any registered valued identification information stored in the data server 514, the method 600 proceeds to block 608 where the authentication server 512 queries the data server 514 as to whether the valued identification has already been read by a valued identification validation unit 502 at the present event. If the valued identification has already been read at the present event the method 600 proceeds to block 610, otherwise, the method 600 proceeds to block 612. In block 612, the authentication server 512 instructs the data server 514 to update the status of the registered valued identification to “read” and register the time that the ticket was read. In block 610, the authentication server 512 queries the data server as to whether a predetermined period of time has expired since the valued identification was first scanned by a valued identification validation unit 502. If the predetermined period of time has expired, the method 600 proceeds to block 611 where the authentication server 512 informs the valued identification validation unit 502, and the valued identification unit 502 informs the ticketholder through the validation indicator, to see an event associate, otherwise, the method proceeds to block 614.

In block 614, the valued identification validation unit 502 informs the ticketholder through the validation indicator that the ticketholders of all tickets registered with the read valued identification information are to proceed to a ticket validation unit 504 at the entrance to the event.

At block 616, the ticketholder presents a ticket to a ticket validation unit 504. The unit 504 reads the ticket information from the ticket and transmits the information to the authentication server 512. The ticket information may comprise ticket identification information, event information, seat information, and other information known o one skilled in the art. In one embodiment, the ticket information consists of ticket identification information containing a unique identifier for each ticket to an event. In block 618, the authentication server 512 queries the data server 514 as to whether the ticket information matches a valid ticket. If the ticket is not valid, the method 600 proceeds to block 620 where the authentication server 512 informs the ticket validation unit 504, and the ticket validation unit 504 informs the operator of the ticket validation unit 504 through the validation indicator, that the ticket is not valid. The ticketholder is then directed to see an event associate for further assistance.

If in block 618 the ticket is valid, the method 600 proceeds to block 622 where the authentication server 512 queries the data server 514 as to whether the ticket has already been used to access the event. If the ticket has already been read by a ticket validation unit 504, the method 600 proceeds to block 624 where the authentication server 512 informs the ticket validation unit 504, and the ticket validation unit 504 informs the operator of the ticket validation unit 504 through the validation indicator, that the ticket has already been used to access the event. The ticketholder is then directed to see an event associate for further assistance.

If in block 622 the ticket has not been used to access the event, the method proceeds to block 626 where the authentication server 512 queries the data server 514 as to whether the registered ticketholder valued identification associated with the ticket has been read by the valued identification validation unit 502. If the registered ticketholder valued identification has not been read, the method 600 proceeds to block 628 where the authentication server 512 informs the ticket validation unit 504, and the ticket validation unit 504 informs the operator of the ticket validation unit 504 through the validation indicator, that the registered ticketholder valued identification has not been read. The ticketholder is then directed to a valued identification validation unit 502.

If in block 626 the registered ticketholder valued identification has been read, the method 600 proceeds to block 634 where the authentication server 512 queries the data server 514 as to whether the registered valued identification associated with the read ticket has been read at a valued identification validation unit 502 within a predetermined period of time. If the predetermined period of time has expired, the method 600 proceeds to block 636 where the authentication server 512 informs the ticket validation unit 504, and the ticket validation unit 504 informs the operator of the ticket validation unit 504 through the validation indicator, the extent to which the predetermine period of time has expired. The operator of the ticket validation unit 504 may then permit the ticketholder to enter the event, proceeding to block 638, or direct the ticketholder to see an event associate. If in block 634 the predetermined period of time has not expired, the method 600 proceeds to block 638 where the ticketholder is granted access to the event and the authentication server 512 instructs the data server 514 to update the status of the ticket as “read”.

As previously described, the system 500 is designed such that the ticket validation unit 504 is located at the entrance to the event while the valued identification validation unit 502 is located at the event away from the entrance to the event. This separation facilitates the efficient validation of event tickets and the entry into the event with minimum delay. Valued identification is validated at a valued identification validation unit 502 prior to the ticketholder arriving at the entrance to the event. Any problems relating to the valued identification are addressed at the valued identification validation unit 502 without delaying ticketholders awaiting entry to the event at the event entrance. In addition, where the same registered ticketholder valued identification information is associated with more than one ticket, the registered ticketholder valued identification is only required to be read one time by a single ticket validation unit 504. Thus, in a group of ticketholders possessing tickets with the same registered ticketholder valued identification information, only one member of the group is required to present the registered ticketholder valued identification at the valued identification validation unit 502 while the remaining members of the group may proceed to the ticket validation unit 504 at the entrance to the event.

In an alternative embodiment, the method 600 may perform a preauthorization of a predetermined amount on the registered ticketholder valued identification associated with a ticket upon the presentation of the registered ticketholder valued identification to the valued identification validation unit 502. In this alternative embodiment, the authentication server 512 communicates the read valued identification information to a valued identification authorization system through a network along with a request for a preauthorization of a predetermined amount. In a preferred alternative embodiment, the predetermined amount is the price of the ticket. If the preauthorization request is denied by the valued identification authorization system, the authentication server 512 directs the data server 504 to mark the registered ticketholder valued identification information as invalid.

In an alternative embodiment, the mobile device 1000 described above may also function as a valued identification validation unit 502. The memory 1006 of the mobile device 1000 contains statements and instructions stored therein that when executed by the processor 1008 provide the following mobile valued identification method. The mobile valued identification method is identical to method 600 with the exception of block 602. In block 602, the ticketholder utilizes their mobile device 1000 to validate their valued identification information instead of a valued identification validation unit 502. The ticketholder utilizes their mobile device 1000 to communicate with the authentication server 512 and request validation of their valued identification information. The ticketholder sends mobile device identification information, positioning information and authentication information to the authentication server 512. The mobile device identification information may comprise a mobile phone number, a mobile PIN, mobile IP address, MAC address, or other identifier for uniquely identifying the ticketholder's mobile device 1000. The positioning information comprises the location of the mobile device 1000 or proximity of the mobile device 1000 to the event as determined by the positioning unit 1012. The authentication information may comprise a user name, password, valued identification information, contact information, or other information for authenticating the ticketholder known to one skilled in the art. The authentication server 512 confirms that the submitted mobile device identification information and authentication information to registered mobile device identification information and authentication information that have been previously registered by the ticketholder with the data server 504 in association with a ticket. In addition, the authentication server 512 confirms that the positioning information is within a predetermined minimum proximity to the event. If the confirmation fails, the authentication server 512 communicates with the mobile device 1000 informing the ticketholder of the failed confirmation. If the confirmation is successful, the authentication server 512 obtains the ticketholder's valued identification information registered with the date server 504 and proceeds to block 604 as described above. In the alternative, the mobile device 1000 prompts the ticketholder to perform the mobile valued identification method described above when the mobile device 1000 is within a predetermined minimum proximity to the event. In the further alternative, the positioning information is not communicated to or utilized by the authentication server. In the further alternative, the ticketholder communicates their valued identification information to the authentication server 512 using their mobile device 1000.

### Secondary Market System

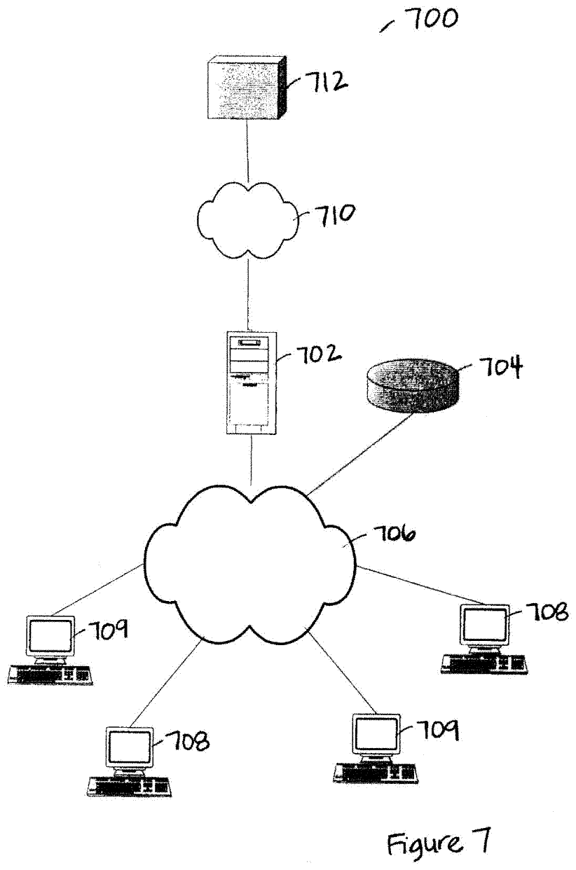

Referring to FIG. 7, a secondary market system 700 is shown. The system 700 enables registered original purchasers of event tickets to resell previously purchased tickets in a secondary market authorized by the original ticket vendors. The system 700 comprises a resale server 702, a data server 704, a network 706, user terminals 708, ticket vendors 709, a network 710, and a valued identification authorization system 712.

The resale server 702 comprises a processor and a computer readable medium (not shown). The computer readable medium contains instructions stored therein that when executed by the processor perform the secondary market method as further described below. The resale server 702 communicates with the data server 704, user terminals 708, and ticket vendors 709 through network 706, and the valued identification authorization system 712 through network 710.

The data server 704 comprises a processor and a computer readable medium (not shown). The computer readable medium contains instructions stored therein that when executed by the processor facilitate the management, communication, access and storage of data on the data server 704. The data consists of information respecting the identification, validity, availability, purchase, bidding, history, ownership and allocation of event tickets. In one embodiment, the data consists of user account information (i.e. user name, password, etc.), the status of each ticket (i.e. purchased, available for purchase, etc.), ticket information (i.e. ticket identification information, event information, seat information, etc.), ownership information (i.e. the original purchaser of each ticket, the ticketholder of each ticket, contact information of the original purchaser, contact information of the ticketholder, etc.), resale restrictions (i.e. tickets not permitted for resale, maximum or minimum resale prices, maximum number of tickets permitted to be resold by each seller, prohibited sellers, permitted sellers, etc.), valued identification information of the ticketholder of each ticket, bid information (i.e. desired event, desired price or price range, number of tickets, desired ticket type and/or location, etc.), and offer information (i.e. event, price or price range, ticket type and/or location, number of tickets, tickets sold together or separately, etc.). The data server 704 communicates with the resale server 702 through network 106. In the alternative, the data server 704 may communicate with the resale server 702 over a separate network from network 706. In the further alternative, the data server 704 may form part of the resale server 702.

In one embodiment, the data server 704 is the same server as data server 104 of system 100. In an alternative embodiment, the data server 104 and data server 704 are separate servers that communicate over a network, and data stored on the data server 104 is copied to the data server 704 at a predetermined frequency.

Users can access the resale server 702 through network 706 via a registration device, such as, user terminals 708 and ticket vendors 709. User terminals 708 may be a computer, cellular phone, personal digital assistant, gaming device or other communication device capable of communicating with a server through a network as known to one skilled in the art. The ticket vendors 709 may be a person or an automated telephone system and users may communicate with the ticket vendors 709 over the phone or in person at designated locations. In one embodiment, the resale server 702 hosts a web site and sellers, purchasers and original ticket vendors communicate with the resale server 702 through the website via a web browser running on the user terminals 708 and ticket vendors 709.

The valued identification authorization system 712 contains information pertaining to valued identification and enables the authorization and charging of monetary amounts to an account associated with valued identification. The valued identification authorization system 712 communicates with the resale server 702 through network 710.

### Secondary Market Method

The system 700 facilitates the secure resale of previously purchased tickets and ensures purchasers are provided with valid tickets and title thereto. The system 700 permits a registered original purchaser (“seller”) to sell a previously purchased ticket by either posting the ticket to the resale server 702 with an associated asking price or offering the ticket to a new purchaser or purchasers that have posted a bid for a ticket on the resale server 702. Similarly, the system 700 permits a new purchaser to purchase a previously purchased ticket by either posting a bid for the ticket to the resale server 702 along with an associated bid price or purchasing a ticket posted to the resale server by a seller.

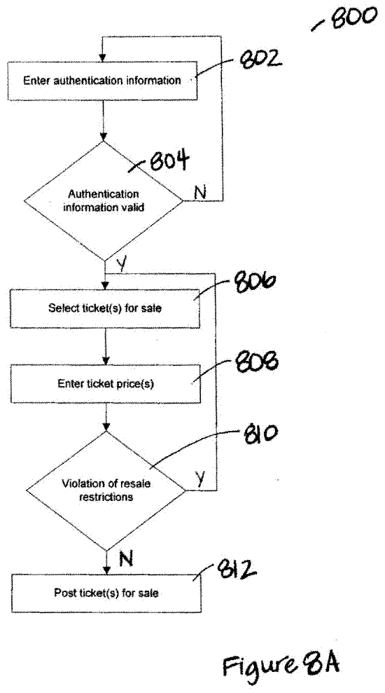

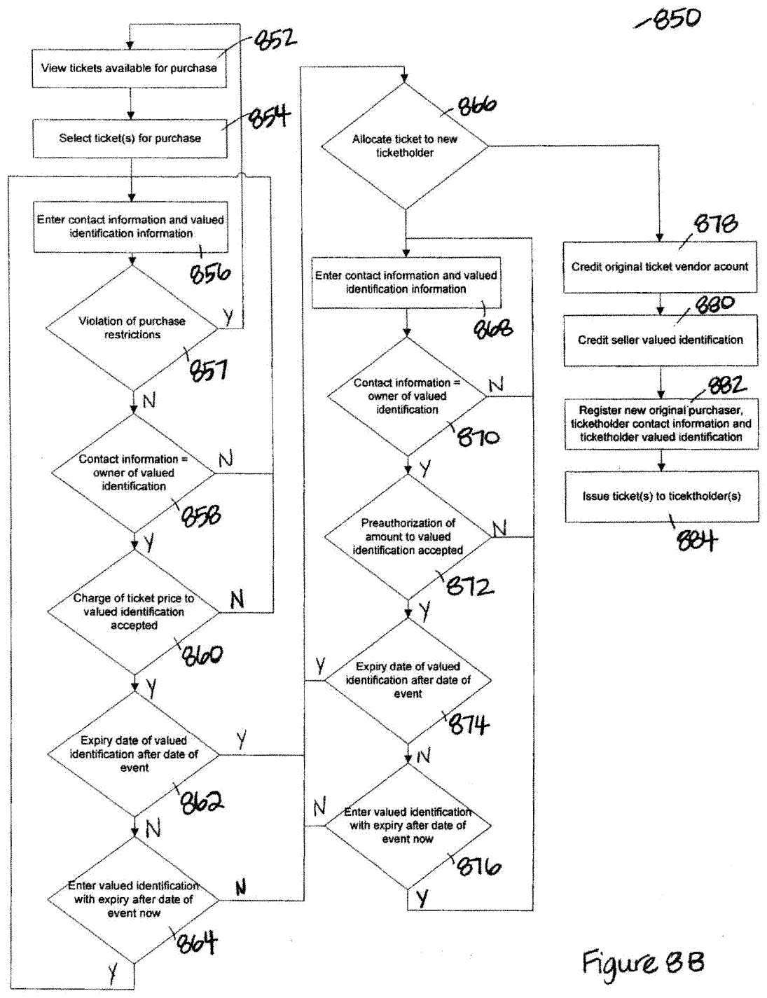

Referring to FIG. 8A, a method 800 of posting one or more previously purchased tickets for sale to the resale server 702 is shown. In block 802, a seller communicates to the resale server 702 through a user terminal 708 or ticket vendor 709 requesting the posting of one or more previously purchased tickets to the resale server 702. The resale server 702 then prompts the seller to enter authentication information to confirm the seller is the registered original purchaser of one or more tickets to the event. The authentication information may comprise one or more of the seller's account information, contact information, original valued identification information, and/or ticket information that has previously been registered with the resale server 702 and/or the ticket server 102 described above. In one embodiment, the authentication information consists of a user name and password. In block 804, the resale server 702 queries the data server 704 to authenticate the authentication information provided by the seller. If the authentication information is invalid the resale server 702 proceeds to block 802, otherwise, the resale server 702 proceeds to block 806.

In block 806, the resale server 702 prompts the seller to select one or more tickets that the seller desires to post for sale on the resale server 702. In block 808, the resale server 702 prompts the seller to enter offer information selected for each ticket selected in block 806. Offer information may comprise the event, price or price range, ticket type and/or location, number of tickets, tickets sold together or separately, and other information pertaining to and offer to sell one or more tickets known to one skilled in the art. In one embodiment, the offer information consists of the price and whether the tickets must be purchased together. In block 810, the resale server 102 queries the data server 704 to determine if the seller has violated any resale restrictions. If the seller has violated any resale restrictions the resale server 702 informs the seller and proceeds to block 806, otherwise, the resale server 702 proceeds to block 812. The resale restrictions may comprise: tickets not permitted for resale, maximum or minimum resale prices, maximum number of tickets permitted to be resold by each seller, prohibited sellers, permitted sellers and other restrictions known to one skilled in the art. In one embodiment, the resale restriction requires that the sale price of each ticket is greater than the original purchase price (i.e. face value) of the ticket.

In block 812, the resale server 702 posts the one or more tickets for sale on the website hosted by the resale server 702. Purchasers may view and purchase the tickets through the resale server 702 as further described below. Alternatively, in block 812, the resale server 702 may communicate offer information respecting the one or more tickets to potential purchasers over any electronic communication medium and in any format known to one skilled in the art, including without limitation, wired or wireless mediums, compressed or uncompressed formats, encrypted or unencrypted formats, email, facsimile, Short Message Service or text messages, Multimedia Messaging Service or multimedia messages, or instant messaging.

Referring to FIG. 8B, a method 850 of purchasing a previously purchased ticket posted on the resale server 102 is shown. In block 852, a purchaser communicates with the resale server 702 through a user terminal 708 or ticket vendor 709 requesting a listing of previously purchased tickets that are posted for sale for a particular event. The resale server 702 queries the data server 704 to acquire the listing and communicates the listing to the user terminal 708 or ticket vendor 709. The listing may be presented based upon the ticket price, original ticket price (i.e. face value), seat location, and/or other offer information. In block 854, the purchaser selects one or more tickets for purchase. If the offer information associate with the tickets requires that multiple tickets must be purchased together, the resale server 702 will only permit the purchaser to select the number of tickets specified. The resale server 702 then holds the selected tickets for a predetermined period during which other purchasers are prohibited from selecting the held tickets for purchase.

Blocks 856 to 876 facilitate the purchase of the tickets by the purchaser and allocation of one or more tickets to one or more new ticketholders if desired by the purchaser. The operation of blocks 856 to 876 are identical to and described in blocks 206 to 226 ticket management method 200, respectively, with the exception that the ticket server 102, data server 104, network 106, user terminals 108, ticket vendors 109, network 110, and valued identification authorization system 112 are replaced with the resale server 702, data server 704, network 706, user terminals 708, ticket vendors 709, network 710, and valued identification authorization system 712, respectively.

In block 878, the resale server 702 credits the original ticket vendor with any service charges, transaction fees, commissions, profits above a certain price of the ticket, and/or other charges designated by the original ticket vendor. In one embodiment, the resale server 702 credits the original ticket vendor with a transaction fee and any profits greater than a certain amount of the original purchase price of the ticket. In the alternative, any of the above fees, charges, commissions, profits, or portions thereof, can be credited to the account of one or more third parties, such as a charity designated by the original ticket vendor and/or seller. In addition, the resale system 702 may automatically issue charitable tax receipts to the seller for amounts credited to the designated charities.

In block 880, the resale system 702, communicates with the valued identification authentication system 712 through network 710 and credits the account associated with the seller's registered valued identification with the purchase price of the tickets less any amounts deducted in block 878. In the alternative, the resale system 702 may deposit the amount in the seller's bank account, issue a cheque to the seller for the amount, register a credit of the amount with the original ticket vendor for future purchases of tickets, goods or services, or provide the amount to the seller in kind or as a credit in other manners known to one skilled in the art.

In block 882, the resale server 702 instructs the data server 704 to update the information associated with the purchased tickets. Specifically, for each purchased ticket the ticket server 702: (a) registers the purchaser as the original purchaser of the ticket; (b) registers the purchaser's contact information and valued identification; and (d) register's the ticketholder's contact information and valued identification information.

In block 884, the resale server 702 reissues the purchased tickets to the registered ticketholder of each ticket. In one embodiment, the ticket is an electronic ticket that is delivered to the registered ticketholder via email, text message, the website hosted by the ticket server 702, or other electronic communication method known to one skilled in the art. Optionally, the electronic tickets may be encrypted, as described above in relation to method 200, such that the ticket cannot be transferred to a third party. In the alternative, the ticket may be a physical ticket that is sent to the registered ticketholder via mail or courier, or picked up by the ticketholder at a specified location.

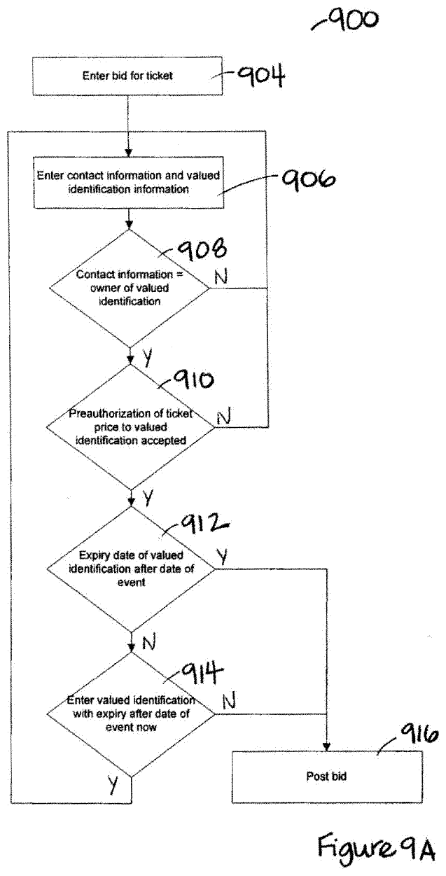

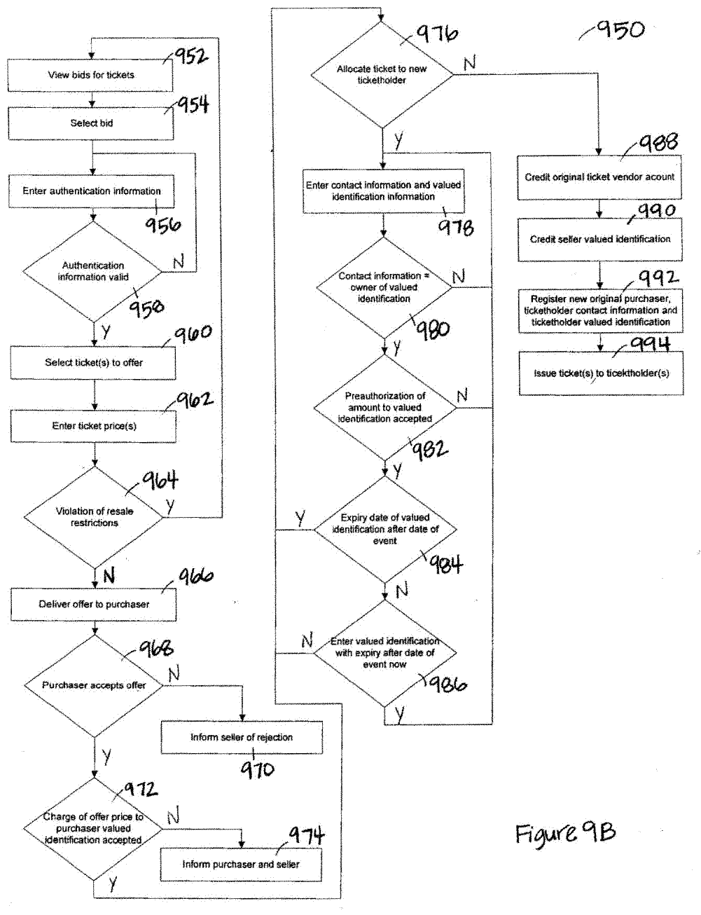

Referring to FIG. 9A, a method 900 of bidding for the purchase of one or more previously purchased tickets using system 700 is shown. In block 904, a purchaser communicates with the resale server 702 through a user terminal 708 or ticket vendor 709 and provides bid information for one or more tickets to an event. The bid information may comprise a desired event, a desired price, price range (i.e. maximum price), or price category (i.e. event seats having a face value in a specified price range), desired areas or seat locations, time limit for a bid, number of tickets, and other information for particularizing a bid for a type of ticket. In one embodiment, the bid information consists of a desired price category, maximum price, and a time limit for which the bid is valid. The desired areas or seat locations may be selected through an interactive graphical event area and seating map made available on the website hosted by the resale server 702.

In block 906, the resale server 702 prompts the purchaser to enter the purchaser's contact information and valued identification information, in the same form as described above in block 206 of method 200. In block 908, the resale server 702 compares the purchaser's contact information to the ownership information of the purchaser's valued identification. If the purchaser's contact information does not match the ownership information of the purchaser's valued identification the resale server 702 informs the purchaser and proceeds to block 906, otherwise, the resale server 702 proceeds to block 910. In block 910, the resale server 702 communicates the purchaser's valued identification information to the valued identification authorization system 712 through network 710 along with a preauthorization request for the price bid for the tickets. If the preauthorization request is denied by the valued identification authorization system 712, the resale server 702 informs the purchaser and proceeds to block 906, otherwise, the resale server 702 proceeds to block 912.

In block 912, the resale server 702 compares the expiry date of the purchaser's valued identification to the date of the event. If the expiry date of the purchaser's valued identification is after the date of the event, the resale server 702 proceeds to block 916, otherwise, the resale server 702 proceeds to block 914. In block 914, the resale server 702 informs the purchaser that prior to the date of the event the purchaser must register valued identification with the resale server 702 having an expiry date after the date of the event. The purchaser is prompted to either enter new valued identification having an expiry date after the date of the event or indicate that the purchaser will enter such valued identification at a later time prior to the date of the event (i.e. by selection of a check box or other manner of indication known to one skilled in the art). If the purchaser selects the entry of new valued identification, the resale server 702 proceeds to block 906, otherwise, the resale server 702 proceeds to block 916.

In block 916, the resale server 702 posts the bid on the website hosted by the resale server 702. Sellers may view the bids and offer previously purchased tickets for sale to bidders through the resale server 702 as further described below. The purchaser may submit one bid or multiple bids for one or more tickets or categories of tickets (i.e. price range, locations, etc.). Alternatively, in block 916, the resale server 702 may communicate the bid to potential purchasers over any electronic communication medium and in any format known to one skilled in the art, including without limitation, wired or wireless mediums, compressed or uncompressed formats, encrypted or unencrypted formats, email, facsimile, Short Message Service or text messages, Multimedia Messaging Service or multimedia messages, or instant messaging.

Referring to FIG. 9B, a method 950 of offering a previously purchased ticket to fulfill a bid for the purchase of a ticket is shown. In block 952, a seller communicates with the resale server 702 through a user terminal 708 or ticket vendor 709 requesting a listing of the bids for tickets for a particular event. The resale server 702 queries the data server 704 to acquire the listing and communicates the listing to the user terminal 708 or ticket vendor 709. In block, 954 the seller selects a bid of a purchaser for which the seller desires to offer one or more of the seller's tickets for sale.

In block 956, the resale server 702 prompts the seller to enter authentication information to confirm the seller is the registered original purchaser of one or more tickets to the event. The authentication information may be one or more of the seller's user account information, original purchaser contact information, original purchaser valued identification information, and/or ticket information that has previously been registered with the resale server 702 and/or the ticket server 102 of system 100 described above. In one embodiment, the authentication information consists of a user name and password. In block 958, the resale server 702 queries the data server 704 to authenticate the authentication information provided by the seller. If the authentication information is not valid the resale server 702 proceeds to block 956, otherwise, the resale server 702 proceeds to block 960.

In block 960, the resale server 702 prompts the seller to select one or more tickets that the seller desires to offer for sale to the purchaser. In block 962, the resale server 702 prompts the seller to enter offer information for each ticket selected in block 960. Offer information may comprise the event, price or price range, ticket type and/or location, number of tickets, tickets sold together or separately, and other information pertaining to and offer to sell one ore more tickets known to one skilled in the art. In one embodiment, the offer information consists of the price and whether the tickets must be purchased together. In block 964, the resale server 102 queries the data server 704 to determine if the seller has violated any resale restrictions, in the same form as described above in block 810 of method 800. If the seller has violated any resale restrictions the resale server 702 informs the seller and proceeds to block 960, otherwise, the resale server 702 proceeds to block 966.

In block 966, the resale server 702 communicates the seller's offer information to the purchaser. In block 968, the purchaser elects to accept the offer or reject the offer by communicating a response to the resale server 702. If the purchaser rejects the offer, the resale server 702 proceeds to block 970 and informs the seller that the offer was rejected. If the purchaser accepts the offer, the resale server 702 proceeds to block 972 and communicates the purchaser's valued identification information to the valued identification authorization system 712 through network 710 along with a request to charge the price of the tickets offered by the seller. If the charge request is denied by the valued identification authorization system 712, the resale server 702 proceeds to block 974 and informs the purchaser and/or seller, otherwise, the resale server 702 proceeds to block 976. Where the seller has made offers for the same tickets to multiple purchasers and one of the purchasers has accepted an offer by the seller, the resale server 702 prevents the other purchasers from selecting the purchase of the tickets in block 968 and sends a communication to the other purchasers informing them that the seller's offer has been withdrawn.

Blocks 976 to 994 permit the purchaser to allocate one or more purchased tickets to one or more new ticketholders, facilitate the crediting of designated portions of the purchase price to the original ticket vendor and/or seller, register the purchaser and new ticketholders in association with the purchased tickets, and reissue the purchased tickets to the new ticketholders. The operation of blocks 976 to 994 are identical to and are described in blocks 866 to 884, respectively, in relation to method 850.

In a further alternative embodiment, in block 884 of method 800 and block 994 of method 900, the registered original purchaser is prompted to select one of the following methods of delivery for the purchased tickets: 1) delivery of the physical tickets by courier or mail; 2) pickup of physical tickets at location, for example, at a will call window; or 3) electronic delivery of electronic tickets. Optionally, the purchaser may select a different method of delivery for each ticket. Where electronic delivery is selected, the resale server 702 will delay electronic delivery of the electronic tickets to the registered ticketholders until a predetermined period before the start of the event, such as, for example, 24 hours prior to the start of the event. In the alternative, the predetermined period may be configure to be any period prior to the start of the event. In the further alternative, the electronic delivery of electronic tickets may be delayed until the valued identification associated with each ticket is subsequently verified at a predetermined minimum period prior to the start of the event as further described below. Amongst other benefits, the delayed delivery of electronic tickets to the predetermined period places an additional impediment to ticketholders attempting to resell their electronic tickets using unauthorized channels.

The resale server 702 also provides historical information of tickets posted for sale, bids for tickets, offers to bids, and completed sales of tickets on the resale server 702.

Once the ticket has be resold through any of processes 800, 850, 900 and 950, the new purchaser and allocated ticketholders may manage the purchased tickets through the ticket management system 100 described above. At any point prior to the completion of the sale, a purchaser may cancel any of the purchaser's bids posted on the ticket server 102 and a seller may cancel any of the seller's offers posted on the ticket server 102.

In an alternative embodiment, the secondary market system 700 may service a plurality of secondary markets for multiple events or the same event. For example, the original ticket vendor may specify a general group of tickets that can be resold in a general secondary market and a specific group of tickets that can only be resold in a specific secondary market, such as, tickets that may be resold only among fan club members, concierge clubs (for example, American Express™ Front of the Line™), or other designated groups.

In a further alternative embodiment, the resale server 702 is configured to present purchasers with the view that the purchaser can expect to see from the seat that the purchaser is considering purchasing. The data server 704 stores a plurality of graphical images (each a “seat view”) depicting views at the event and registers the seat views with the associated ticket for the event. In block 852 of method 800 and block 966 of method 966, the resale server 702 queries the data server 704 to determine if a seat view is associated with the ticket. If a seat view is available, the resale server 702 communicates the seat view, or link to the seat view, to the purchaser. Before purchasing the ticket, the purchaser may select the seat view associated with the ticket to get a sense of the view from the seat or area for which the ticket relates. In one embodiment, the same seat view may be used for a predefined group of tickets. For example, the tickets relating to a block of seats at an event could have the same associated seat view representative of the view from the center seat of the block of seats. In the alternative, the seat view representative of the view from any location within the block of seats may be associated with all of the tickets in the block of seats. In the further alternative, a distinct seat view may be association with each ticket. In a further alternative embodiment, the resale server 702 permits a user access the seat view of tickets listed in the history of tickets sold through the system 700.

In a further alternative embodiment, a seller may request the resale server 702 to automatically send updates to the seller reporting new high bids posted to the system. Similarly, a purchaser may request the resale server 702 to automatically send updates to the purchaser reporting new low offers posted to the system. The seller and purchaser may request the ticket server 702 to restrict the updates to report bids and offers respecting tickets in a particular category, such as, price category, location of associated seat at the event, or other criteria know to one skilled in the art. The updates may also comprise information on previous bids and offers, the next highest or lowest bids and offers, ticket information, purchase restrictions, and other relevant information to the ticket.

In a further alternative embodiment, in block 812 the resale server 702 determines if the one or more previously purchased tickets posted for sale to the resale server 702 satisfy the bid information of any of the bids submitted to the resale server 702 in accordance with method 900. For each bid that is satisfied by one or more of the tickets posted for sale, the resale server 702 communicates to the purchaser submitting the bid the particulars of those tickets. The purchaser may then purchase the tickets in accordance with method 850. Similarly, in block 916 the resale server 702 determines if one or more tickets posted for sale to the resale server 702 satisfy the bid information of the bid submitted by the purchaser. For each of the one or more tickets that satisfy the bid, the resale server 702 communicates to the purchaser submitting the bid the particulars of those tickets. The purchaser may then purchase the tickets in accordance with method 850.

In a further alternative embodiment, the system 700 may permit a third party agent, such as a concierge, to purchase tickets on behalf of a purchaser. The third party agent purchase tickets in the identical manner described above except in block 856 of method 850 and block 906 of method 900, the resale server 702 permits the third party agent to enter account information that uniquely identifies the third party agent. All ticket purchases for which the third party agent participates using the system 700 are recorder on the data server 704. The original ticket vendor may then offer various forms of compensation to the third party agent depending on the number or value of ticket purchases for which the agent participates.

In a further alternative embodiment, the system 500 and system 700 are configured to permit a ticketholder in attendance at an event (“segmented event”) comprising multiple games, acts, parts or segments (each an “event segment”) to leave the segmented event, resell tickets to one or more of the remaining event segments of the segmented event to one or more purchasers, and authenticate the resold tickets to the event segments.

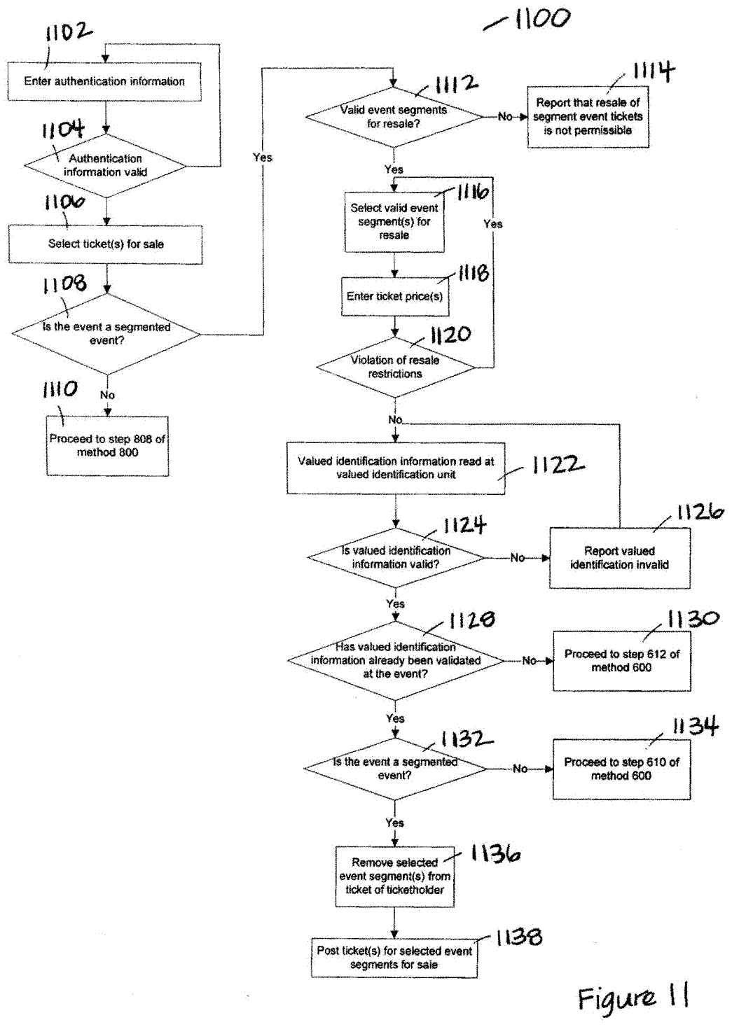

Referring to FIG. 11 a method 1100 of offering for resale one or more tickets to one or more events segments of a segmented event is shown. In blocks 1102 to 1106 a seller communicates with the resale server 702 through a user terminal 708 or ticket vendor 709 and selects one or more tickets to the event that the seller desires to resell. Blocks 1102 to 1106 are identical to blocks 802 to 806 of method 800. These blocks may be performed during (a) the purchase of the tickets by the seller through the ticket management system 100 or the secondary market system 700, or (b) after the purchase of the tickets by the seller either prior to or during a segmented event.

In block 1108, the resale server 702 queries the data server 704 to determine if the event associated with the selected tickets is a segmented event. If the event is a segmented event, the method 1100 proceeds to block 1112, otherwise, the method 1100 proceeds to block 1110 where the method 1100 proceeds block 808 of method 800 and continues with method 800 until completion.

In block 1112, the resale server 702 queries the data server 704 to determine if there are any valid event segments for which the selected tickets are permitted to be resold. If none of the event segments are valid, the method 1100 proceeds to block 1114 where the resale server 702 informs the seller that resale of tickets for the segmented event is not permitted. However, if any of the event segments are valid, the method 1100 proceeds to block 1116. The validation of the event segments comprises determining which event segments have not completed, whether the seller is authorized to resell tickets to the event segments, and whether the ticket is authorized for resale (for example, complimentary tickets may be precluded from resale).

In block 1116 the resale server 702 communicates to the seller the valid event segments that the seller is permitted to resell and prompts the seller to select the event segments for which the seller desires to resell tickets. Once the seller selects the event segments, the method 1100 proceeds to block 1118 where the resale server 702 prompts the seller to enter offer information respecting the sale of tickets for the selected event segments. Offer information may comprise the event segment, price or price range, ticket type and/or location, number of tickets, tickets sold together or separately, and other information pertaining to and offer to sell one or more tickets known to one skilled in the art. In one embodiment, the offer information consists of the price and whether the tickets must be purchased together.

The method 1100 then proceeds to block 1120 where the resale server 702 queries the data server 704 to determine if the seller has violated any resale restrictions. If the seller has violated any resale restrictions the resale server 702 informs the seller of the violation and proceeds to block 1116, otherwise, the resale server 702 proceeds to block 1122. The resale restrictions may comprise: tickets not permitted for resale, maximum or minimum resale prices, maximum number of tickets permitted to be resold by each seller, prohibited sellers, permitted sellers and other restrictions known to one skilled in the art. In one embodiment, the resale restriction requires that the sale price of each ticket is greater than the original purchase price (i.e. face value) of the ticket prorated by the number of event segments in the event.

In block 1122, the seller presents valued identification to the valued identification validation unit 502. The unit 502 reads valued identification information from the valued identification and transmits the information to the authentication server 512. The method 1100 then proceeds to block 1124 where the authentication server 512 queries the data server 514 to match the read valued identification information to registered valued identification information stored in the data server 514. If the read valued identification information does not match any registered valued identification information stored in the data server 514, the method 1100 proceeds to block 1126, where the authentication server 512 informs the seller through the valued identification validation unit 502 that the read valued identification is not valid. The method 1100 then proceeds to block 1122.

If in block 1124 the read valued identification information matches any registered valued identification information stored in the data server 514, the method 1100 proceeds to block 1128 where the authentication server 512 queries the data server 514 as to whether the valued identification has already been validated at the present event in accordance with method 600. If the valued identification has already been validated at the present event the method 1100 proceeds to block 1132, otherwise, the method 1100 proceeds to block 1130 where the method 1100 proceeds to block 612 of method 600 and continues with method 600 until completion.

In block 1132, the authentication server 512 queries the data server 514 to determine if the current event is a segmented event. If the current event is a segmented event, the method 1100 proceeds to block 1136, otherwise, the method 1100 proceeds to block 1134 where the method 1100 proceeds to block 610 of method 600 and continues with method 600 until completion.

In block 1136, the resale server 702 communicates with the data server 704 to cancel the seller's ticket such that the seller is no longer permitted to access the event with his or her ticket. The method 1100 then proceeds to block 1138 where the resale server 702 posts the one or more tickets for the selected event segments for sale on the website hosted by the resale server 702. Purchasers may view and purchase the tickets through the resale server 702 in the manner described above in method 850. A purchaser of the tickets for any of the selected event segments may then use the ticket to access the event for the event segment in the manner described above in method 600. Alternatively, in block 1138, the resale server 702 may communicate offer information respecting tickets to the selected event segments to potential purchasers over any electronic communication medium and in any format known to one skilled in the art, including without limitation, wired or wireless mediums, compressed or uncompressed formats, encrypted or unencrypted formats, email, facsimile, Short Message Service or text messages, Multimedia Messaging Service or multimedia messages, or instant messaging.

In the alternative, in block 1136 the resale server 702 does not cancel the seller's ticket, instead, the resale server 702 communicates with the data server 704 to remove the selected event segments for resale from the ticket information associated with the seller's ticket such that the seller can no use the ticket to access the event for the selected event segments, however, the seller may use the ticket to access the event for any remaining event segments that have not been selected for resale. In the further alternative, block 1136 occurs after block 1138, such that the seller's ticket or the selected event segments are not cancelled or removed from the ticket information associated with the seller's ticket until one or more tickets to the selected event segments are resold to a purchaser.

Optionally, in order to assist in ensuring that an individual entering the segmented event with an event ticket exits the event prior to the resale of event segments under the ticket, the seller may be required to allocate the event tickets to the individuals that will be using the tickets to enter the segmented event when (a) the seller purchases the tickets or (b) after the purchase of the tickets, the seller indicates a desire to resell event segments under the tickets. In this manner, a ticket to event segments of a segmented event cannot be resold until the valued identification of the individual attending the event is presented at a valued identification validation unit 502 outside of the segmented event.

In the further alternative, at block 1118 of method 1100, the seller may indicate that the seller will enter a ticket price for the tickets each segmented event at a later time. The method 1100 will delay the posting tickets to the selected event segments for resale until the seller has provided valid ticket prices in accordance with blocks 1118 and 1120 of method 1100. For example, during purchase of event tickets to segmented event, the seller may setup selected event segments for resale upon the departure of the seller from the segmented event, however, the seller may desire to defer specifying a resale price for the tickets until the seller has the opportunity to assess the secondary market for the tickets either prior to or during the event.

In a further alternative embodiment, instead of presenting valued identification to a valued identification unit 502 in block 1124, the seller utilizes his or her mobile device 1000 to initiate the resale of selected event segments. Specifically, in block 1124 the seller sends mobile device identification information, positioning information and authentication information to the authentication server 512 upon exiting the event. The mobile device identification information may comprise a mobile phone number, a mobile PIN, mobile IP address, MAC address, or other identifier for uniquely identifying the ticketholder's mobile device 1000. The positioning information comprises the location of the mobile device 1000 or proximity of the mobile device 1000 to the event as determined by the positioning unit 1012. The authentication information may comprise a user name, password, valued identification information, contact information, or other information for authenticating the ticketholder known to one skilled in the art.

The authentication server 512 confirms that the submitted mobile device identification information and authentication information to registered mobile device identification information and authentication information that have been previously registered by the ticketholder with the data server 504 in association with a ticket. In addition, the authentication server 512 confirms that the positioning information indicates that the seller has exited the event. If the confirmation fails, the authentication server 512 communicates with the mobile device 1000 informing the seller of the failed confirmation. If the confirmation is successful, the authentication server 512 obtains the seller's valued identification information registered with the date server 504 and proceeds to block 1128.

In the alternative, the mobile device 1000 prompts the ticketholder to perform the mobile valued identification method described above when the mobile device 1000 is within a predetermined minimum proximity to the event. In the further alternative, the positioning information is not communicated to or utilized by the authentication server. In the further alternative, the ticketholder communicates their valued identification information to the authentication server 512 using their mobile device 1000.

### Biometric Identification

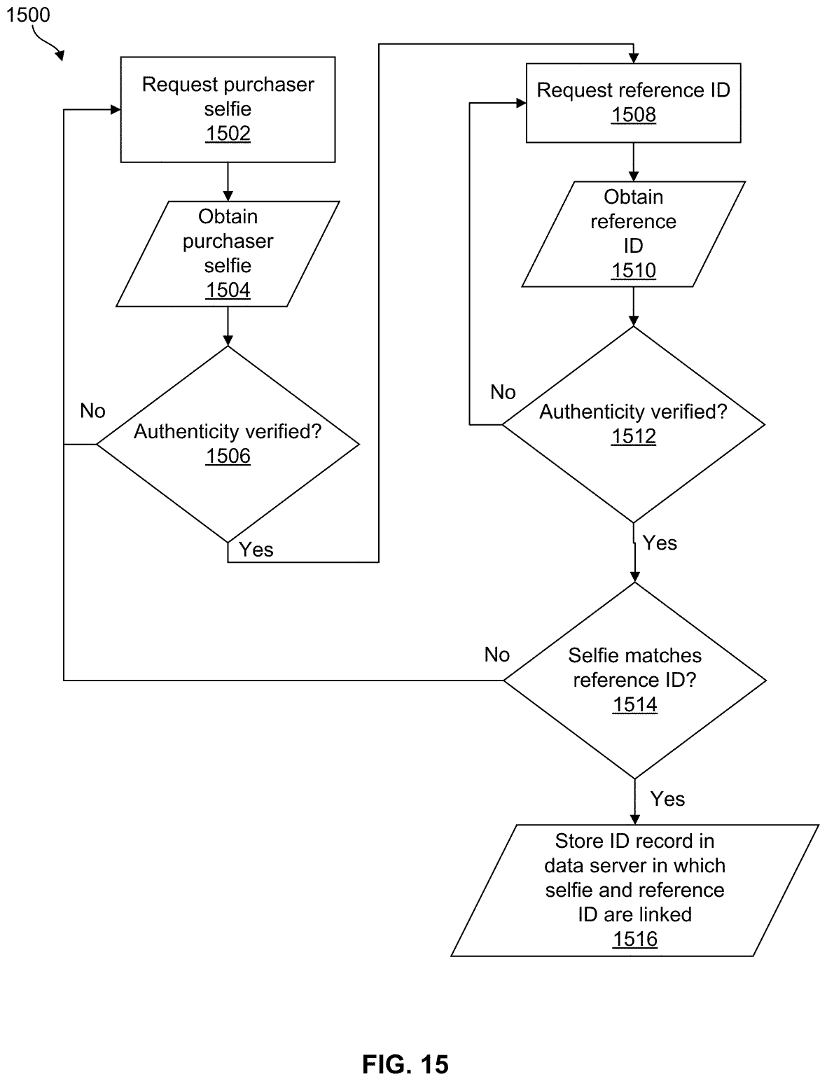

In at least some example embodiments, identification information for a ticketholder need not be valued identification information. Rather, the identification information may comprise a type of biometric identification that uses, for example, one or more of a ticketholder's face, voice, palm print and fingerprints for identity verification purposes. This is depicted in FIG. 15, which is a process diagram of an example method of generating and storing an identification record in the data server 104. The identification record can then be used to verify the identity of a ticketholder. In at least some embodiments, the method may be applied to the system 100 as depicted in FIG. 1. In at least some other embodiments, including the embodiment discussed in more detail below, the system 100 of FIG. 1 may be varied to include a biometric identification (ID) server (not depicted) communicatively coupled to the network 106, in a manner analogous to the ticket server 102 and data server 104. As discussed further below, the biometric ID server may be used to store biometric information for all users, with the information for only a subset of users attending the event transferred to the ticket server 102 and/or data server 104 for data security purposes. In other embodiments, two servers could be employed—a “master server” (where biometric information is initially captured and stored, e.g., upon purchase or transfer) and a “venue server” (which downloads the biometric information from the venue server as needed for a particular event, or in some cases stored for recurring or other future events at that same venue).

Referring now to block 1502, the system 100 requests that the purchaser supply a selfie: i.e., a photo that captures the purchaser's face. In at least some embodiments, the system 100 makes this request via an application running on one of the user terminals 108, such as the purchaser's phone, and requires the purchaser to actually take a photo as opposed to use a photo stored in a photo library. This ensures the photo is of a person actually present at the user terminal 108, helping to mitigate against fraud. The photo may alternatively be taken or otherwise input to the system from one of the ticket vendors 109. For example, one of the ticket vendors 109 may capture the biometrics of the ticketholder in person prior to the event, such as when a season ticket holder picks up paper tickets in person from the vendor.

The purchaser supplies the selfie at block 1504, and the system 100 performs liveness detection on the photo at block 1506 to ensure the photo is of a live person as opposed to, for example, a printout of a person. Liveness detection may be performed on device at, for example, the ticket vendor 109 or user terminal 108; alternatively, the selfie may be transmitted to the biometric ID server and the liveness check may be performed by the biometric ID server. The liveness detection may be based, for example, on depth sensor data captured with the visible image data of the selfie. The authenticity check may also comprise determining whether the selfie has sufficient detail to permit generation of a feature vector of the purchaser's face that can subsequently be used to confirm the purchaser's identity, as discussed further below. If selfie authenticity is not verified, the system 100 rejects the selfie and returns to block 1502 where it requests another selfie from the purchaser.

If selfie authenticity is verified, the system 100 proceeds to block 1508 where it requests a reference photo identification (ID) from the purchaser. The reference photo ID may be, for example, a suitable form of government issued ID such as a driver's license, service card, or passport. At block 1510, the purchaser provides an image of the reference photo ID. This may be done by photographing the reference photo ID at the time the request of block 1508 is made; alternatively, since “liveness” is not an issue with a reference photo ID, in at least some embodiments the system 100 may permit the purchaser to select an image of the reference photo ID from a photo library.

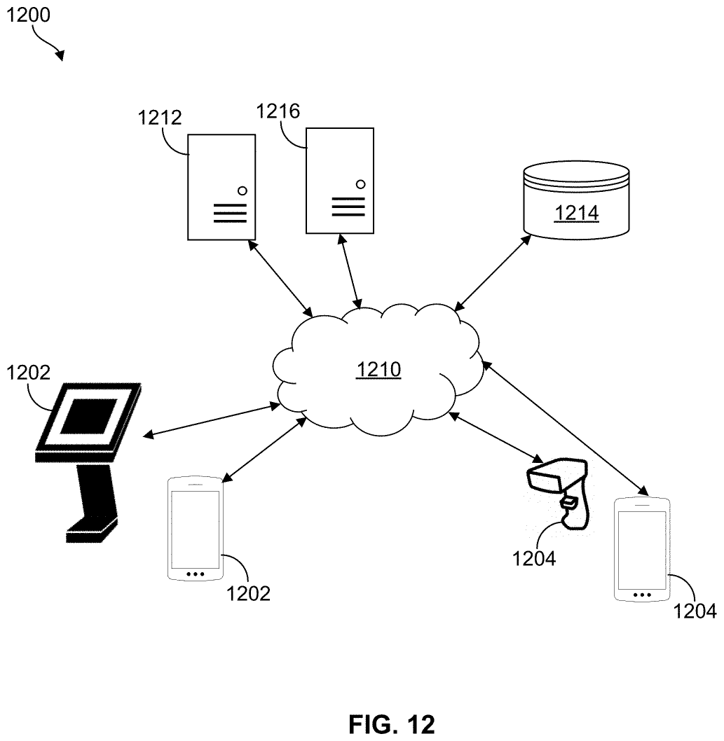

After obtaining the image of the reference photo ID, the system 100 at block 1512 checks to see whether the image of the reference photo ID holder's face, which is part of the larger image of the entire reference photo ID, has been tampered with, using any suitable tamper detection method. If the authenticity of the reference photo ID is rejected, the system 100 returns to block 1508 and again requests an image of a reference photo ID from the purchaser. As at block 1506, authenticity may be verified, for example, by the ticket server 102, user terminal 108, biometric ID server, and/or ticket vendor 109. If the authenticity of the reference photo ID is accepted, the system 100 proceeds to block 1514 and compares the purchaser's selfie to the reference photo ID to ensure they identify the same individual. For example, the ticket server 102 may determine feature vectors based on faces identified in the selfie and reference photo ID, determine the Euclidean distance between the two feature vectors, and if the distance satisfies a similarity threshold then determine that the selfie and reference photo ID depict the same person, thereby confirming the purchaser's identity. If the system 100 confirms the purchaser's identity, at block 516 the system 100 stores in the data server 104 an ID record in which the purchaser's selfie is linked to the reference photo ID and in which a feature vector determined from the purchaser's face as depicted in one or both of the selfie and reference photo ID is used as a reference for identity verification during ticket validation, as described above for FIGS. 5 and 6 and below for FIGS. 12-14. The feature vector used for subsequent verification may be the feature vector determined from the selfie, from the reference photo ID, or a combination thereof (e.g., an average of the two).

In at least some embodiments (not depicted), the method 1500 may further comprise obtaining additional data from the reference ID over and above the reference photo ID holder's face. For example, the ticket server 102 may additionally scrape information such as the reference photo ID holder's name and birthdate and store this information in the data server 1214. As discussed in further detail below, the system 1200 may use this data to query the third party server 1216 to determine the health or vaccination status of the reference photo ID holder.

While FIG. 15 contemplates using facial recognition, as mentioned above the system 100 may additionally or alternatively use other forms of biometric identification (proxy identities) based on, for example, the ticketholder's voice, fingerprints, and/or palm prints. This may be mandatory or the system 100 may provide the purchaser with an option to use one of multiple forms of biometric identification (in addition to 2FA policies that require multiple different forms of identification, biometric or otherwise). For example, in addition to or as an alternative to a selfie, the system 100 may request that the purchaser recite a number of phrases from which the ticket server 102 generates an audio fingerprint for the purchaser analogous to the facial feature vector described above. As another example, the system 100 may additionally or alternatively rely on a retinal photo. As another example, the system 100 may additionally or alternatively obtain fingerprint information from the purchaser. This may be done, for example, by obtaining a purchaser's fingerprint using a fingerprint sensor at one of the user terminals 108 (e.g., via a mobile phone) or one of the ticket vendors 109. The device used to obtain the fingerprint may be any suitable device, such as a laptop or other mobile device, or an auxiliary self-enrollment station. Corresponding audio fingerprints or physical fingerprints identifying the purchaser may comprise part of the reference photo ID (e.g., the fingerprints may be stored on a magnetic strip on government issued ID). As with the facial biometric information, in at least some example embodiments any non-facial biometric information is stored in the biometric ID server and shared with the ticket server 102 only as necessary. In at least some example embodiments, the system 1200 requires the purchaser or ticketholder to first confirm their identity using facial identification, and following that initial confirmation to switch to an alternative type of biometric identification such as voice, palm print, and/or fingerprint identification. This allows an event holder to keep facial biometric information saved as a reference in the biometric ID server, and a ticketholder to be able to validate their tickets using a device that is only able to perform identity verification using a different type of biometric data. For example, the purchaser may have their facial biometric information taken by the ticket vendor 109 at time of purchase, but not own a device that is capable of capturing a facial image suitable for biometric identification (e.g., the ticketholder's mobile device may be a smart phone without a depth sensor, which in some embodiments is required for the liveness check). The ticketholder may accordingly have to rely on voice recognition and/or fingerprint recognition for ticket validation, assuming the ticketholder's mobile device has a microphone and/or a fingerprint scanner. Permitting a different type of biometric identification additionally permits facial biometric information to remain on the biometric ID server while sharing only non-facial and potentially less sensitive biometric information, such as audio information, to the ticket server 102.

While in at least some embodiments the facial biometric information is stored in the biometric ID server through at least until the end of ticket validation, in at least some other example embodiments the facial biometric information is stored only as necessary to verify another form of biometric identification, such as voice, palm print, and/or fingerprint identification. For example, in the example above in which the system 1200 requires the purchaser or ticketholder to first confirm their identity using facial identification, and following that initial confirmation the purchaser or ticketholder switches to an alternative type of biometric identification, the system 1200 may delete the facial biometric information immediately or shortly after it has been used to permit the purchaser or ticketholder to switch to another form of biometric identification. Each time a purchaser purchases a ticket, they may rely on facial biometric information for identity verification if such information is stored in the biometric ID server, or alternatively may proceed through the method 1500 of FIG. 15 if the facial biometric information had been deleted and is not available to the system 1200.

During ticket validation as discussed above in FIGS. 5 and 6, the type of biometric information collected by the system 500 at the event may comprise reciting a pre-recorded voice message on a mobile device or at one of the ticket validation units 504, using either a mobile device or ticket validation unit 504 to receive a live face selfie or using a mobile device or ticket validation unit 504 to verify other biometrics such as retina or finger/thumbprint. If validation fails, the purchaser is notified in at least some embodiments that they have a deadline by which to meet the identity verification requirements or the ticket will be void for entry into the event. However, in at least some embodiments the ticketholder may transfer or sell the ticket to another person via the secondary market, as discussed above, who can still use the ticket to be admitted to the event if they satisfy the identity verification requirements. In at least some example embodiments, the ticketholder may fall back on valued identification, such as a credit card, in the event biometric identification fails during ticket activation.

In at least some example embodiments, biometric facial identification may be used concurrently with one or more other forms of identification, whether those other forms of identification are biometric information, valued identification information, or another type of identification information. For example, in some cases the system 1200 (e.g., via the authentication server 1212) may be unable to definitively conclude that a person attempting to verify their identity at one of the identification verification units 1202 is the purchaser/ticketholder whose biometrics are stored in the biometric ID server. For example, the authentication server 1212 may require that the ticketholder's identity be verified such that the likelihood of a false positive identification verification is less than a false positive maximum threshold. However, using facial biometric information alone, the authentication server 1212 may only be able to verify the ticketholder's identity such that the likelihood of a false positive is above the maximum false positive threshold. However, when the facial biometric information is used in conjunction with another type of identification information, such as an alternative type of biometric identification information (e.g., an audio recording), valued identification information (e.g. a credit card), and/or non-valued and non-biometric identification information (e.g., a driver's license), the authentication server 1212 may be able to verify the ticketholder's identity with a certainty such that the risk of a false positive verification is less than the maximum false positive threshold. For example, the authentication server 1212 may require there to be a less than 1/1,000 chance of a false positive identification verification in order for identity to be verified, and using facial biometrics alone may only be able to verify identity to within 1 in 500. However, facial biometrics combined with an audio sample may permit the authentication server 1212 to verify the ticketholder's identity with a less than 1/1,500 chance of a false positive verification. In an example such as this, the authentication server 1212 is able to verify the identity of a ticketholder using multiple forms of identification information in a case where facial biometric information alone would have been insufficient. In at least some other embodiments, the presence or use of any complementary or secondary form of identification may be sufficient for identity verification or ticket validation regardless of the results of the attempted facial biometrics identification (e.g., if identify can be definitively verified via a secondary form of identification such as the ticketholder's voice or fingerprint, that is sufficient to verify the identity of the ticketholder regardless of the results of a facial scan of the ticketholder).

In at least some example embodiments, in addition to relying on biometric identification to identify a ticketholder in the process of admitting the ticketholder to the event, the system 500 may use biometric identification to identify a ticketholder in the process of admitting them to different monitored areas within a venue at which the event is hosted. In order to gain access to each of the monitored areas, the system 500 further comprises additional identification verification units 1202 (each an “internal identification verification unit 1202”) respectively associated with each of the monitored areas at the venue. Each of the monitored areas is cordoned off such that to gain entrance to one of the monitored areas, the ticketholder verifies their identity at the internal identification verification unit 1202 before being permitted to enter the monitored area. In at least some embodiments, the ticketholder also verifies their identity at the same or another internal identification verification unit 1202 when leaving the monitored area, thereby providing the system 1200 with a time window during which the ticketholder is confirmed to be within the monitored area. Example monitored areas comprise VIP, lounge, and/or backstage areas; and stores and kiosks for food, beverage, and merchandise purchases. Tracking identities of ticketholders as they enter, and in some embodiments as they leave, various monitored areas may be useful, such as for disease contact tracing, in the event the location of a particular ticketholder at a particular time is desired to be known within an event.

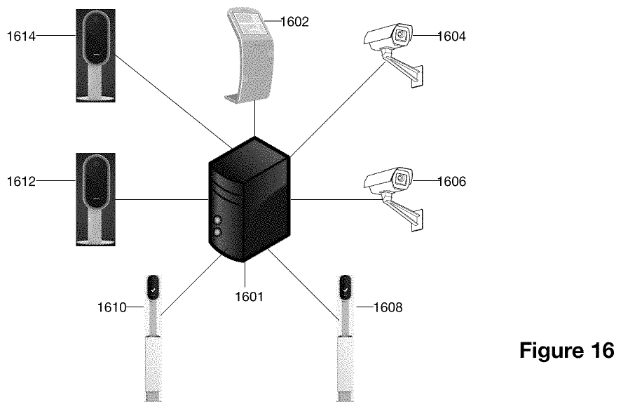

FIG. 16 depicts example devices networked to an example biometric server 1601 used to monitor when the ticketholder is present at various monitored areas, according to another example embodiment. In FIG. 16, each of the following is networked to a data server 1601: a self serve food & beverage kiosk 1602; a camera 1604 monitoring entry to a private suite; a camera 1606 monitoring a box office pick-up window; a ticket scanner 1608 for controlling access to certain venue areas, such as VIP, lounge, and suite floor areas; a ticket scanner 1610 for controlling backstage entry; a ticket scanning station 1612 for food and beverage pick-up; and a ticket scanning station 1614 for merchandise pick-up. When a ticketholder interacts with any of these devices, the time of the interaction and the identity of the ticketholder who interacted are recorded by the data server 1601, providing information for use during subsequent contact tracing.

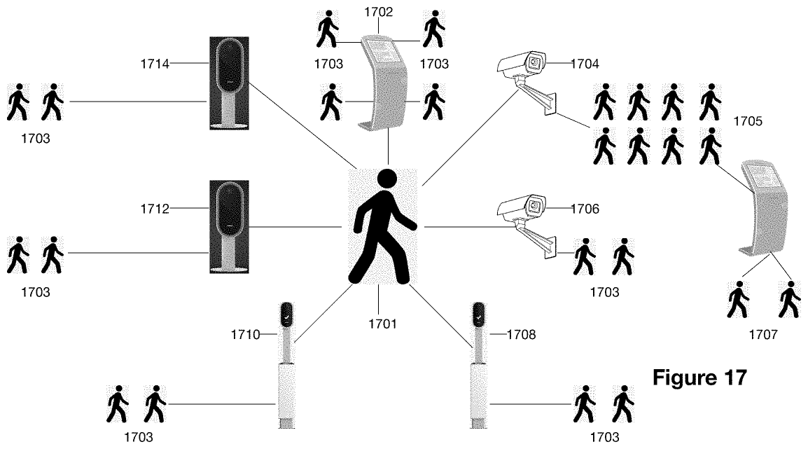

FIG. 17 depicts example relationships between a particular ticketholder 1701 being contact traced by virtue of their identity and location being recorded at any one or more of a self serve food & beverage kiosk 1702; a camera 1704 monitoring entry to a private suite; a camera 1706 monitoring a box office pick-up window; a ticket scanner 1708 for controlling access to certain venue areas, such as VIP, lounge, and suite floor areas; a ticket scanner 1710 for controlling backstage entry; a ticket scanning station 1712 for food and beverage pick-up; and a ticket scanning station 1714 for merchandise pick-up, respectively corresponding to the kiosk 1602, camera 1604, camera 1606, scanner 1608, scanner 1610, station 1612, and station 1614 of FIG. 16. As discussed above in respect of FIG. 16, the system 500 maintains a record of the ticketholder 1701's interactions with each of the devices shown in FIG. 17, as well as interactions other groups 1703, 1705, 1707 of attendees have with those devices. In FIG. 17, all of the groups 1703, 1705 had interactions with those devices at the same or at approximately the same time as the ticketholder 1701. For example, at the time the ticketholder 1701 is recorded at the kiosk 1702, camera 1706, scanners 1708, 1710, and stations 1712, 1714, the group 1703 of attendees are other attendees who were standing in front, behind, or next to the ticketholder 1701. For the camera 1706, facial recognition may be used to identify the attendees near the ticketholder 1701 when the ticketholder 1701 is also recorded by the camera 1706. For the kiosk 1702, scanners 1708, 1710, and stations 1712, 1714, the interactions the attendees of the group 1703 had with the same devices a predetermined time before and after the ticketholder 1701 are deemed to be those attendees who were potentially exposed and who are to be contact traced. FIG. 17 also shows another kiosk associated with two groups 1705, 1707 of attendees, to which analogous contact tracing principles are applied.

### Confirming One or More Health Criteria Prior to Event Admittance

Events, particularly indoor events with thousands of attendees, may be curtailed during a pandemic involving transmission of a communicable disease. While certain events may be prohibited during the peak of a pandemic, as the pandemic wanes public health authorities may permit certain types of events to again be held. For example, as people develop disease immunity such as through vaccination or by virtue of having caught and recovered from the disease, those immune persons may safely attend large indoor and outdoor events. However, tracking thousands of event attendees to ensure they have a sufficient level of disease immunity, or satisfy one or more other health criteria, is technically problematic. Doing so requires correlating each ticketholder's health information with their ticket accurately, maintaining that correlation despite the fact that health information may change after ticket purchase and before the event, all in a manner that mitigates against the risk of circumvention.

The embodiments as depicted in FIGS. 12 to 14 are directed at confirming that a ticketholder satisfies one or more health criteria (e.g., stored as a digital vaccine passport or QR code) before permitting that ticketholder to be admitted to an event. In FIG. 12, a system 1200 comprises a network 1210 to which an authentication server 1212, data server 1214, third party server 1216, verification units 1202 depicted as a mobile device and a kiosk, and ticket validation units 1204 depicted as a mobile device and a handheld scanner are communicatively coupled. The network 1210, authentication server 1212, data server 1214, identification validation units 1202, and ticket validation units 1204 are respectively analogous to the network 510, authentication server 512, data server 514, valued identification validation units 502, and ticket validation units 504 described above in respect of FIG. 5, except as otherwise noted herein. The verification units 1202, for example, differ from the valued identification validation units 502 as follows:
1. In at least some example embodiments, the verification units 1202 are not limited to using valued identification. Rather, they may use any suitable form of identification, even if that identification cannot be charged a value for the purchase of goods and services. In addition to valued identification, example suitable identification comprises a driver's license, services card, and biometric identification as described above.
2. The verification units 1202 may also be used to enter a ticketholder's health data into the system 1200, which health data is required for ticket authentication to proceed, as described further below.

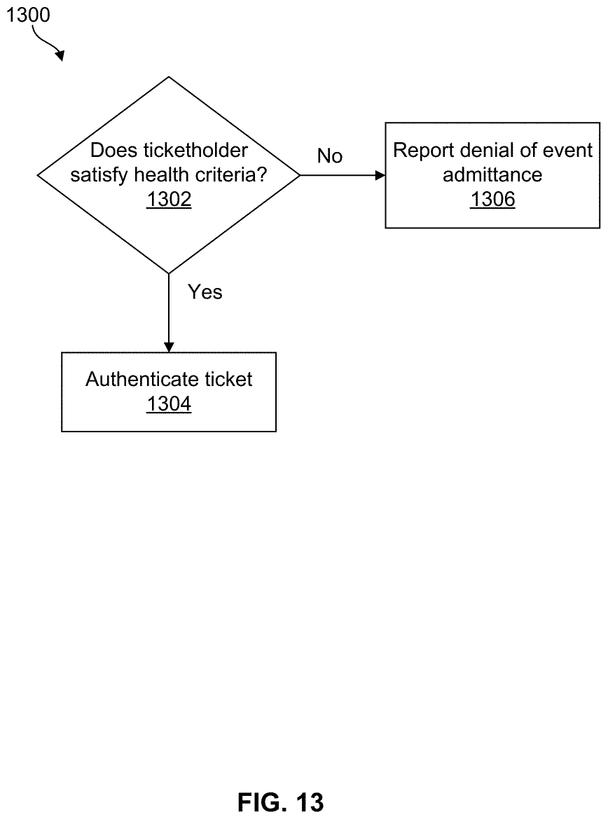

The system 1200 is used to perform a method 1300 for ticket authentication as depicted in FIG. 13. The method 1300 comprises confirming, by one or more servers and in advance of the event, that the ticketholder satisfies one or more health criteria (block 1302). If the system 1200 cannot make this confirmation, it reports a denial of event admittance (block 1306). For example, the authentication server 1212 may determine that admittance is to be denied and communicate this denial to the ticket validation unit 1204. An attendant using the ticket validation unit 1204 at the entrance to the event may then refuse the ticketholder admittance.

If the system 1200 confirms that the ticketholder satisfies the health criteria, it may then proceed to continue the ticket authentication process (block 1304). This process is analogous to the method 600 of FIG. 6, as described above. For example, after confirming that the ticketholder satisfies the one or more health criteria, the system 1200 reads current identification information, at a location away from an entrance of the event, that identifies the ticketholder (block 602); after the current identification information is read, communicates the current identification information to one or more servers, such as the authentication server 1212 and/or the data server 1214; reads ticket information from a ticket held by the ticketholder at the entrance of the event (block 616); after the ticket information is read, communicates the ticket information to the one or more servers; and determines that the ticketholder may be granted access to the event after determining that the ticket information is valid (block 618) and the current identification information matches registered identification information registered with the one or more servers before the current identification information is read (block 604). The one or more servers may determine that the ticket information is valid and the current identification information matches the registered identification information, and then communicate to the entrance of the event, such as to someone using one of the ticket validation units 1204, that the ticketholder may be granted access to the event. In at least some example embodiments, “current identification information” comprises valued identification information and additional types of identification information, such as biometric identification and a driver's license as described above. In other example embodiments, “current identification information” may be a subset of this; for example, it may comprise only biometric identification and valued identification information, or it may comprise only valued identification information.

Confirming, by one or more servers and in advance of the event, that the ticketholder satisfies one or more health criteria may take a variety of forms. In the presently described embodiment, the actual act of determining may be performed by the authentication server 1212 based on a confirmation retrieved from the data server 1214 by the authentication server 1212. In some other embodiments, the functionality may be performed by different servers. For example, the authentication server 1212 and data server 1214 may be combined into a single server that performs the above-described functionality. As another example, the authentication server 1212 may access the third party server 1216, owned or controlled by a health authority (e.g., one that is, or that is controlled directly or indirectly by, a governmental authority), to obtain the confirmation that the ticketholder satisfies the one or more health criteria. As mentioned above, the system 1200 may earlier have scraped from the reference photo ID personal information such as the name and birthdate of the ticket purchaser. This information may be sent to the third party server 1216, and in response the third party server 1216 may send a confirmation in the form of binary yes/no response. The third party server 1216 may also provide a health-related confirmation by acting as a hub that collects data from one or more other servers (not depicted) that provide health-related confirmations, and based on the confirmations from those one or more other servers return an appropriate confirmation to the authentication server 1212. For example, the third party server 1216 may query servers owned or controlled by health authorities representing a variety of states or provinces and, if each of those servers confirms that a particular ticketholder satisfies one or more health criteria, return a single confirmation back to the authentication server 1212. The third party server 1216 may cache results obtained from third party servers to reduce the number of times the third party server 1216 queries other servers connected to it; in at least some embodiments, cached results may periodically expire, in which case the third party server 1216 would re-query servers as necessary to obtain new confirmation(s).

In the presently described example embodiment, the one or more health criteria comprise that the ticketholder is uninfected with a communicable disease, such as COVID-19. The fact that the ticketholder is uninfected may be evidenced in any one or more ways, such as by evidence of vaccination, antibodies, and/or a negative test for the disease within a certain period of time prior to acquiring the ticket or the date of the event. In at least some embodiments, the health criteria may not be a simple binary metric; instead, they may represent different degrees of public health risk. For example, a ticketholder who has a negative test for the disease the same day as the event may be given a low risk rating; a different ticketholder whose most recent negative test is a week before the event may be given a moderate risk rating; another ticketholder whose most recent negative test is two weeks before the event may given a high risk rating; and ticketholders whose most recent negative test if more than two weeks before the event may be treated the same as ticketholders who have not received any negative test. A user of the system 1200 may decide to admit all ticketholders who satisfy a minimum risk level and treat them identically. Alternatively, a user of the system 1200 may assign different conditions to the tickets of ticketholders of different risk levels. For example, ticketholders in identical risk categories may be assembled or seated together at the event.

The confirmation that the ticketholder does satisfy the one or more health criteria may be entered into the system 1200, and more particularly into the data server 1214, in any one or more suitable ways. In at least some example embodiments, the data server 1214 stores a list of acceptable data sources, such as certain government agencies (e.g., public health authorities or government ministries), and those agencies are permitted to upload data directly to the data server 1214 via an appropriate interface (e.g., via a web service or application programming interface). For example, for each ticketholder, an agency may provide a YES or NO token representing whether the ticketholder satisfies the one or more health criteria (e.g., the authentication server 1212 sends to the data server 1214 information on the ticketholder's name, birthdate, medical ID numbers and vaccination status (including, for example vaccination dose dates, from which expiration dates could be inferred), and in response the data server 1214 returns the YES or NO token confirming or refuting the information contained in the server's 1212 query). In this way, the system 1200 can receive a confirmation that the ticketholder satisfies the one or more health criteria, and store the confirmation in the data server 1214.

As another example, the system 1200 may receive data access credentials and, using the data access credentials, retrieve a confirmation that the ticketholder satisfies the one or more health criteria from the third party data source. For example, the data access credentials may comprise a link (e.g., a uniform resource locator) to a third party website that provides confirmation; for security purposes, the link may in some embodiments comprise a randomly generated string of characters. Alternatively, the system 1200 may receive from the ticketholder login credentials to a third party data source, such as a website or computer application, and, using the login credentials, retrieve a confirmation that the ticketholder satisfies the one or more health criteria from the third party data source. For example, the login credentials may take the form of a username and/or registration number permit the authentication server 1212 to access the ticketholder's lab results evidencing a negative test for a communicable disease. The authentication server 1212 may log into the third party data source, obtain the confirmation in the form of the negative test, and store the confirmation in the data server 1214.

As another example, instead of providing data access credentials, the ticketholder may upload to the system 1200 an image of a card, certificate, or screen (e.g., a digital vaccine passport or QR code) identifying the ticketholder and confirming that the ticketholder satisfies the one or more health criteria. The image comprises computer-recognizable text and/or images identifying the ticketholder and confirming that the ticketholder satisfies the one or more health criteria. The authentication server 1212 automatically scans the text and/or images, confirms that the identification information in the image identifies the ticketholder by comparing the identification information to the identification information the ticketholder registered with the system 1200 at the time of acquiring the ticket, confirms what the health information in the image represents, and stores in the data server 1214 a data structure indexed by ticketholder representing the health information.

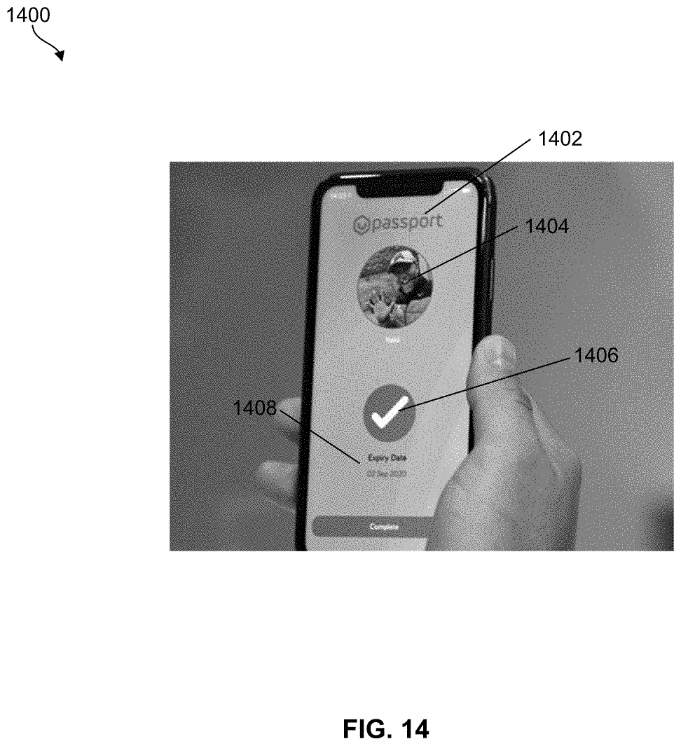

FIG. 14 depicts an example image 1400 that the ticketholder may upload to the authentication server 1212, and that the authentication server 1212 may process as described above. The image 1400 comprises an information source identifier 1402, in this case in the form of the PASSPORT′ design; an image 1404 of the ticketholder's face, which the authentication server 1212 processes and uses to confirm that the health information in the image 1400 is that of the ticketholder, such as by determining and then comparing the feature vector of the face in the image with feature vector stored in the data server 1214, as described above; the health information 1406 itself, which in this case is in the form of a large checkmark confirming that the ticketholder has satisfied the testing administered by the source of the health information; and an expiry date 1408 identifying when the health information expires. As part of confirming the ticketholder satisfies the health criteria, the authentication server 1212 confirms that the expiry date 1408 is after the date the event for which the ticketholder has a ticket occurs.

In at least some example embodiments, the confirmation that the ticketholder receives may be conditional. For example, in the case of a communicable disease, an unconditional confirmation may be vaccination from or antibodies to the disease, while a conditional confirmation may be a negative test result for the disease. The negative test result is conditional because the ticketholder may be infected with the disease subsequent to obtaining the negative test result. Evidence of such possible infection may be a result of contact tracing persons who have come into contact with someone confirmed to have the disease. To account for this possibility, the system 1200 may check one or more times after issuing the conditional confirmation to see if the ticketholder's health condition has potentially changed.

The system 1200 may check multiple times in a manner analogous to how it obtained the conditional confirmation. For example, if the authentication server 1212 logged into a health authority to obtain the conditional confirmation, it may again log into the health authority to confirm the conditional confirmation should not be revoked. Alternatively, the system 1200 may check multiple times in different ways. For example, the authentication server 1212 may obtain the conditional confirmation by logging into a health authority, and may obtain a subsequent confirmation by asking the ticketholder to scan a vaccination card. As discussed above, the authentication server 1212 may obtain confirmation (conditional or otherwise, including confirmations based on antibodies or vaccination) from the third party server 1216; and, the third party server 1216 may be standalone or act as a hub that marshals confirmations from one or more additional servers, such as servers owned or controlled by provincial or state health authorities.

If the system 1200 determines that the ticketholder's health has potentially changed and that the ticketholder no longer satisfies the one or more health criteria, the authentication server 1212 revokes the initial confirmation. This prevents the ticketholder from subsequently authenticating their ticket. Additionally, if the ticket has already been validated but the ticketholder has not yet been admitted to the event, the authentication server 1212 may invalidate the ticket such that the ticket will not show as valid when validation is attempted using one of the ticket validation units 1204.

If sufficient time remains between when the initial confirmation is revoked and the start of the event, the ticketholder may attempt to be re-confirmed by obtaining a subsequent confirmation that the ticketholder satisfies the one or more health criteria. For example, in the case when the system 1200 grants an initial confirmation based on a negative test result and revokes the initial confirmation based on contact tracing, the system 1200 may re-confirm the ticketholder in response to the ticketholder showing evidence of a negative test result obtained more than a disease incubation period after the contact tracing. For all conditional confirmations, the system 1200 may do a subsequent check to ensure the conditional confirmation remains valid a certain time before the start of the event (e.g., 1 day or 1 hour before the start of the event).

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

The terminology used herein is for the purpose of describing particular embodiments only and is not intended to be limiting. Accordingly, as used herein, the singular forms “a”, “an”, and “the” are intended to include the plural forms as well, unless the context clearly indicates otherwise. It will be further understood that the terms “comprises” and “comprising”, when used in this specification, specify the presence of one or more stated features, integers, steps, operations, elements, and components, but do not preclude the presence or addition of one or more other features, integers, steps, operations, elements, components, and groups. Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. The term “and/or” as used herein in conjunction with a list means any one or more items from that list. For example, “A, B, and/or C” means “any one or more of A, B, and C”.

A “computer” or “server” used in the foregoing embodiments may comprise, for example, a processing unit (such as a processor, microprocessor, or programmable logic controller, including when they form part of a central processing unit or graphical processing unit) communicatively coupled to a non-transitory computer readable medium having stored on it program code for execution by the processing unit, microcontroller (which comprises both a processing unit and a non-transitory computer readable medium), field programmable gate array (FPGA), system-on-a-chip (SoC), an application-specific integrated circuit (ASIC), or an artificial intelligence accelerator. Examples of computer readable media are non-transitory and include disc-based media such as CD-ROMs and DVDs, magnetic media such as hard drives and other forms of magnetic disk storage, semiconductor based media such as flash media, random access memory (including DRAM and SRAM), and read only memory. In at least some example embodiments, a computer may also be embedded in or otherwise comprise part of a device such as a smartphone, tablet, television set, holographic projector, headset, and other similar or analogous devices.

It is contemplated that any part of any aspect or embodiment discussed in this specification can be implemented or combined with any part of any other aspect or embodiment discussed in this specification.

In construing the claims, it is to be understood that the use of computer equipment, such as a processor, to implement the embodiments described herein is essential at least where the presence or use of that computer equipment is positively recited in the claims.

One or more example embodiments have been described by way of illustration only. This description is being presented for purposes of illustration and description, but is not intended to be exhaustive or limited to the form disclosed. It will be apparent to persons skilled in the art that a number of variations and modifications can be made without departing from the scope of the claims.

1. A method comprising:
(a) confirming, by one or more servers and in advance of an event, that a ticketholder of the event satisfies one or more health criteria;
(b) after confirming that the ticketholder satisfies the one or more health criteria:
(i) reading current identification information, at a location away from an entrance of an event, that identifies the ticketholder;
(ii) after the current identification information is read, communicating the current identification information to the one or more servers;
(iii) reading ticket information from a ticket held by the ticketholder at the entrance of the event;
(iv) after the ticket information is read, communicating the ticket information to the one or more servers; and
(v) determining that the ticketholder may be granted access to the event after determining that:
(1) the ticket information is valid; and
(2) the current identification information matches registered identification information registered with the one or more servers before the current identification information is read.

2. The method of claim 1, wherein the one or more health criteria comprise that the ticketholder is uninfected with a communicable disease.

3. The method of claim 1 or 2, wherein confirming that the ticketholder satisfies the one or more health criteria comprises:
(a) obtaining a conditional confirmation that the ticketholder satisfies the one or more health criteria;
(b) revoking the conditional confirmation; and
(c) obtaining a final confirmation that the ticketholder satisfies the one or more health criteria.

4. The method of claim 3, wherein the initial confirmation is revoked in response to the ticketholder being contact traced after exposure to the communicable disease.

5. The method of any one of claims 1 to 4, wherein the one or more servers determine that the ticket information is valid and the current identification information matches the registered identification information, and further comprising communicating from the one or more servers to the entrance of the event that the ticketholder may be granted access to the event.

6. The method of any one of claims 1 to 5, wherein confirming that the ticketholder satisfies the one or more health criteria comprises confirming an identify of the ticketholder against the registered identification information.

7. The method of any one of claims 1 to 6, wherein confirming that the ticketholder satisfies the one or more health criteria comprises retrieving from a database a confirmation that the ticketholder satisfies the one or more health criteria.

8. The method of claim 7, wherein confirming that the ticketholder satisfies the one or more health criteria further comprises:
(a) receiving a confirmation that the ticketholder satisfies the one or more health criteria from a health authority; and
(b) storing the confirmation in the database.

9. The method of claim 8, further comprising scanning personal information identifying the ticketholder from an identification card identifying the ticketholder, wherein the personal information comprises part of a query to the health authority.

10. The method of claim 9, wherein scanning the personal information comprises scanning text on the identification card.

11. The method of claim 9, wherein scanning the personal information comprises scanning a photo of the ticketholder on the identification card.

12. The method of claim 7, wherein confirming that the ticketholder satisfies the one or more health criteria further comprises obtaining an image of a card, certificate, or screen identifying the ticketholder and confirming that the ticketholder satisfies the one or more health criteria.

13. The method of claim 7, wherein confirming that the ticketholder satisfies the one or more health criteria further comprises:
(a) receiving data access credentials to a third party data source; and
(b) using the data access credentials, retrieving a confirmation that the ticketholder satisfies the one or more health criteria from the third party data source.

14. The method of claim 13, wherein the data access credentials comprise a link to the third party data source.

15. The method of claim 13, wherein the data access credentials comprise login credentials to a data server.

16. The method of any one of claims 1 to 15, wherein the current identification information is read on a mobile device or a kiosk.

17. A system comprising:
(a) a identification validation unit for placement at an event away from an entrance of the event, the identification validation unit comprising a first processor, an identification reader that is communicative with the first processor, and a first computer readable medium that is communicative with the first processor and that comprises instructions that when executed by the first processor cause the identification unit to read current identification information from identification presented to the valued identification reader and to communicate the current identification information to one or more servers;
(b) a ticket validation unit for placement at the entrance of the event, the ticket validation unit comprising a second processor, a ticket reader that is communicative with the second processor, and a second computer readable medium that is communicative with the second processor and that comprises instructions that when executed by the second processor cause the ticket validation unit to read ticket information from a ticket presented to the ticket reader and to communicate the ticket information to the one or more servers;
(c) wherein the one or more servers are configured to:
(i) confirm that a ticketholder of the event satisfies one or more health criteria; and
(ii) after confirming that the ticketholder satisfies the one or more health criteria:
(1) receive the current identification information from the identification validation unit;
(2) receive the ticket information from the ticket validation unit; and
(3) determine that the ticketholder may be granted access to the event after determining that:
a) the ticket information is valid; and
b) the current identification information matches registered identification information registered with the one or more servers before the current identification information is read.

18. The system of claim 17, wherein the one or more health criteria comprise that the ticketholder is uninfected with a communicable disease.

19. The system of claim 17 or 18, wherein confirming that the ticketholder satisfies the one or more health criteria comprises the one or more servers:
(a) obtaining a conditional confirmation that the ticketholder satisfies the one or more health criteria;
(b) revoking the conditional confirmation; and
(c) obtaining a final confirmation that the ticketholder satisfies the one or more health criteria.

20. The system of claim 19, wherein the initial confirmation is revoked in response to the ticketholder being contact traced after exposure to the communicable disease.

21. The system of any one of claims 17 to 20, wherein the one or more servers:
(a) determine that the ticket information is valid and the current identification information matches the registered identification information; and
(b) communicate to the ticket validation unit that the ticketholder may be granted access to the event.

22. The system of any one of claims 17 to 21, wherein confirming that the ticketholder satisfies the one or more health criteria comprises confirming an identify of the ticketholder against the registered identification information.

23. The system of any one of claims 17 to 22, wherein confirming that the ticketholder satisfies the one or more health criteria comprises retrieving from a database a confirmation that the ticketholder satisfies the one or more health criteria.

24. The system of claim 23, wherein confirming that the ticketholder satisfies the one or more health criteria further comprises:
(a) receiving a confirmation that the ticketholder satisfies the one or more health criteria from a health authority; and
(b) storing the confirmation in the database.

25. The system of claim 23, wherein confirming that the ticketholder satisfies the one or more health criteria further comprises obtaining an image of a card, certificate, or screen identifying the ticketholder and confirming that the ticketholder satisfies the one or more health criteria.

26. The system of claim 23, wherein confirming that the ticketholder satisfies the one or more health criteria further comprises:
(a) receiving data access credentials to a third party data source; and
(b) using the data access credentials, retrieving a confirmation that the ticketholder satisfies the one or more health criteria from the third party data source.

27. The system of claim 26, wherein the data access credentials comprise a link to the third party data source.

28. The system of claim 26, wherein the data access credentials comprise login credentials to a data server.

29. The system of any one of claims 17 to 28, wherein the identification validation unit comprises a mobile device or a kiosk.

30. A non-transitory computer readable medium having program code stored thereon, wherein the program code is executable by a processor and, when executed by the processor, causes the processor to perform the method of any one of claims 1 to 16.

31. A method comprising:
(a) obtaining a first picture of a face of a ticketholder;
(b) obtaining a reference photo identification of the ticketholder, wherein the reference photo identification comprises a second picture of the ticketholder and confirms an identity of the ticketholder;
(c) confirming that the first picture and the second picture are both of the ticketholder;
(d) generating an identification record linking the ticketholder to the identity confirmed by the reference photo identification; and
(e) storing the identification record in a database.

32. The method of claim 31, further comprising:
(a) receiving biometric information identifying the ticketholder of an event by face;
(b) retrieving the identification record from the database;
(c) confirming the biometric information matches the identification record; and
(d) after confirming the biometric information matches the identification record, admitting the ticketholder to the event.

33. The method of claim 31, further comprising obtaining a non-facial biometric identifier identifying the ticketholder after confirming that the first picture and the second picture are both of the ticketholder, wherein the identification record comprises the non-facial biometric identifier.

34. The method of claim 33, further comprising:
(a) receiving non-facial biometric information identifying the ticketholder;
(b) retrieving the identification record from the database;
(c) confirming the non-facial biometric information matches the non-facial biometric identifier comprising part of the identification record; and
(d) after confirming the biometric information matches the identification record, admitting the ticketholder to the event.

35. The method of any one of claims 31 to 34, further comprising:
(a) obtaining from the reference photo identification non-photo personal information identifying the ticketholder; and
(b) storing as part of the identification record the non-photo personal information.

36. The method of claim 35, further comprising querying a third party data server using the non-photo personal information to determine a health status of the ticketholder.

37. A method comprising:
(a) reading current identification information, at a location away from an entrance of an event, that identifies the ticketholder, wherein the current identification information comprises biometric facial information of the ticketholder;
(b) after the current identification information is read, communicating the current identification information to the one or more servers;
(c) reading ticket information from a ticket held by the ticketholder at the entrance of the event;
(d) after the ticket information is read, communicating the ticket information to the one or more servers; and
(e) determining that the ticketholder may be granted access to the event after determining that:
(i) the ticket information is valid; and
(ii) the current identification information matches registered identification information registered with the one or more servers before the current identification information is read.

38. The method of claim 37, wherein the current identification information further comprises complementary identification information also identifying the ticketholder, and wherein determining that the current identification information matches the registered identification information comprises using both the biometric facial information and the complementary identification information to identity verification.

39. The method of claim 38, wherein the complementary identification information comprises non-facial biometric information.

40. The method of claim 38, wherein the complementary identification information comprises non-biometric information.

41. The method of any one of claims 37 to 40, wherein the event is held at a venue, and wherein access to different areas of the venue is monitored using devices for identifying or recording identities of event attendees in the respective different areas, and further comprising storing identification information of the attendees in the respective different areas that is obtained using the respective devices.

42. A system comprising:
(a) a identification validation unit for placement at an event away from an entrance of the event, the identification validation unit comprising a first processor, an identification reader that is communicative with the first processor, and a first computer readable medium that is communicative with the first processor and that comprises instructions that when executed by the first processor cause the identification unit to read current identification information from identification presented to the valued identification reader and to communicate the current identification information to one or more servers;
(b) a ticket validation unit for placement at the entrance of the event, the ticket validation unit comprising a second processor, a ticket reader that is communicative with the second processor, and a second computer readable medium that is communicative with the second processor and that comprises instructions that when executed by the second processor cause the ticket validation unit to read ticket information from a ticket presented to the ticket reader and to communicate the ticket information to the one or more servers;
(c) wherein the one or more servers are configured to:
(i) receive the current identification information from the identification validation unit, wherein the current identification information comprises biometric facial information of the ticketholder;
(ii) receive the ticket information from the ticket validation unit; and
(iii) determine that the ticketholder may be granted access to the event after determining that:
(1) the ticket information is valid; and
(2) the current identification information matches registered identification information registered with the one or more servers before the current identification information is read.

43. The system of claim 42, wherein the current identification information further comprises complementary identification information also identifying the ticketholder, and wherein determining that the current identification information matches the registered identification information comprises using both the biometric facial information and the complementary identification information to identity verification.

44. The system of claim 43, wherein the complementary identification information comprises non-facial biometric information.

45. The system of claim 43, wherein the complementary identification information comprises non-biometric information.

46. The system of any one of claims 43 to 45, wherein the event is held at a venue divided into different areas, and further comprising respective devices positioned to monitor the different areas and for identifying or recording identities of event attendees in the respective different areas, and wherein the one or more servers are further configured to store identification information of the attendees in the respective different areas that is obtained using the respective devices.

47. A non-transitory computer readable medium having program code stored thereon, wherein the program code is executable by a processor and, when executed by the processor, causes the processor to perform the method of any one of claims 31 to 40.
